(19) United States
(12) Patent Application Publication
(10) Pub. No.: US 2021/0169848 A1
(43) Pub. Date: Jun. 10, 2021

(54) USE OF NEUROKININ-1 ANTAGONISTS TO TREAT PRURITUS

(71) Applicant: VYNE THERAPEUTICS INC., Bridgewater, NJ (US)

(72) Inventors: Steven BASTA, Bridgewater, NJ (US); Paul KWON, Bridgewater, NJ (US)

(21) Appl. No.: 17/263,491

(22) PCT Filed: Jul. 26, 2019
(86) PCT No.: PCT/US19/43658

§ 371 Date: Jan. 26, 2021

Related U.S. Application Data
(60) Provisional application No. 62/711,176, filed on Jul. 27, 2018.

(51) Int. Cl. A61K 31/403; A61P 17/04; A61K 9/00

(52) U.S. Cl. CPC A61K 31/403; A61K 9/0053; A61P 17/04

(57) ABSTRACT

The disclosure relates to the use of neurokinin-1 (NK-1) antagonists in treating pruritus in a subject, wherein i) the duration of the pruritus is about one year or longer, ii) the subject is about 40 years of age or older: iii) the subject has no inflammatory skin disease; and/or iv) the pruritus is idiopathic.

### CROSS REFERENCE TO RELATED APPLICATION

This application claims priority to and the benefit of U.S. Provisional Patent Application No. 62/711,176, filed on Jul. 27, 2018, the entire disclosure of which is incorporated herein by reference in its entirety.

### TECHNICAL FIELD

The present disclosure relates to the use of a neurokinin-1 (NK-1) antagonist in treating pruritus in patients at least 40 years of age, having the disease for one year or longer, having no inflammatory skin disease, and/or having idiopathic pruritus.

### BACKGROUND OF THE DISCLOSURE

Pruritus (itch) is an unpleasant sensation that provokes a desire to scratch. Pruritus can have its origin directly in the skin or can develop in the central nervous system (CNS) via hematogenic or neurogenic mediators. Chronic pruritus (such as pruritus lasting≥one year) is a common symptom of skin disorders as well as a wide range of systemic, neurological and psychiatric disorders in the absence of a skin condition, and can be induced by many different types of medications. The prevalence of chronic pruritus in the general adult population is about 14-17% and increases with age, with about 60% of the elderly (over 65 years of age) suffering from moderate to severe chronic pruritus. Chronic pruritus can be intense, intractable and debilitating, can increase the severity of the underlying disease, and can greatly diminish the quality of life, with many patients suffering from insomnia, anxiety and depression. Stress and anxiety can be induced by the constant pruritus, and stress and anxiety increase the intensity and frequency of the itch, leading to a vicious cycle that affects patient behavior (e.g., scratching) and worsens disease prognosis and quality of life. Persistent rubbing or scratching can form secondary skin lesions such as excoriations, erosions, eschars, hyperpigmentation or patches of discoloration, impetiginisations and scars. Pruritus can induce an itch/scratch cycle and self-stimulation of the pruritic mechanism and scratching, which can exacerbate existing skin lesions and create new skin lesions. Chronic scratching worsens symptoms and often produces open skin lesions, which are susceptible to secondary infections, scarring and potential disfigurement. Once the itch/scratch cycle becomes established, it can be very difficult to stop, thereby perpetuating pruritus.

### SUMMARY OF THE DISCLOSURE

The present disclosure provides for the use of an antagonist (or inhibitor) of neurokinin-1 (NK-1) in treating pruritus in subjects at least 18 years of age, having the disease for six months or longer, having no inflammatory skin disease, and/or having idiopathic pruritus. In certain embodiments, the pruritus has a duration of at least about 6 months, 1 year, 2 years, 3 years or 5 years. In some embodiments, the subject is about 40 years of age or older. In some embodiments, the subject has no inflammatory skin diseases. In certain embodiments, the NK-1 antagonist is serlopitant or a pharmaceutically acceptable salt, solvate, hydrate, clathrate, polymorph, prodrug, metabolite or stereoisomer thereof. In some embodiments, the pruritus is refractory or resistant to other antipruritic therapies without an NK-1 antagonist. In some embodiments, the pruritus is idiopathic. In some embodiments, the pruritus is of unknown origin. In some embodiments, the pruritus is chronic. In some embodiments, the pruritus is chronic pruritus of unknown origin. In some embodiments, the subject has no inflammatory skin disease at the time of administration of the NK-1 antagonist. In some embodiments, the subject has no history of inflammatory skin disease. In some embodiments, the subject has no inflammatory skin disease at the time of administration of the NK-1 antagonist but has a history of inflammatory skin disease. In some embodiments, the subject has no inflammatory skin disease at the time of administration of the NK-1 antagonist and has no history of inflammatory skin disease.

In some embodiments, the subject is about 40 years of age or older, has no inflammatory skin disease, and the duration of the pruitus is about one year or longer and is idiopathic. In some embodiments, the subject is about 40 years of age or older, has no inflammatory skin disease, and the duration of the pruritus is about one year or longer and is of unknown origin.

In some embodiments, the therapeutically effective amount (e.g., per day or per dose) of the NK-1 antagonist for treating pruritus is about 0.25 or 1 to 200 mg, 0.5 or 1 to 150 mg, 0.5 or 1 to 100 mg, 0.5 or 1 to 50 mg, 0.5 or 1 to 10 mg, 10-20 mg, 20-30 mg, 30-40 mg, 40-50 mg, 50-100 mg, 100-150 mg or 150-200 mg. In further embodiments, the therapeutically effective amount of the NK-1 antagonist is administered one or more times a day, once every two days, once every three days, twice a week or once a week (e.g., once or twice daily). In some embodiments, the therapeutically effective amount of the NK-1 antagonist is about 0.25 or 1 to 5 mg or 5-10 mg, or about 0.5 mg, 1 mg, 5 mg or 10 mg, once or twice daily. In certain embodiments, the therapeutically effective amount of the NK-1 antagonist is about 5 mg once daily. In certain embodiments, treatment of pruritus with the NK-1 antagonist lasts for at least about 1 week, 2 weeks, 3 weeks, 4 weeks, 5 weeks, 6 weeks, 2 months, 3 months, 4 months, 5 months, 6 months, 1 year, 2 years, 3 years, 4 years, 5 years or longer (e.g., at least about 1 week, 6 weeks, 3 months, 6 months or 1 year). The NK-1 antagonist can also be taken in an irregular manner or pro re nata (as needed), as described elsewhere herein.

### BRIEF DESCRIPTION OF THE DRAWINGS

A better understanding of features and advantages of the present disclosure will be obtained by reference to the following detailed description, which sets forth illustrative embodiments of the disclosure, and the accompanying drawings.

FIG. 1 shows change in AI-NRS and WI-NRS in subjects having pruritus for one year or longer following 8-week treatment with serlopitant in comparison to the placebo group.

FIG. 2 shows percentage change in AI-VAS 4-Point Responder Rate in subjects with and without inflammatory skin diseases following 6-week treatment with serlopitant (5 mg) or placebo.

FIG. 3 shows percentage change in WI-NRS 4-Point Responder Rate in subjects with known or unknown cause of PN following 8-week treatment with serlopitant (5 mg) or placebo.

FIGS. 4A-4B shows change in AI-VAS from baseline in subjects with various stinging (A) or burning (B) conditions following 8-week treatment with serlopitant (5 mg) or placebo.

FIG. 5 shows percentage change in NRS 4-Point Responder Rate in subjects with and without inflammatory skin diseases in four testing groups: placebo, 0.25 mg, 1 mg, and 5 mg of serlopitant, following 6-week treatment.

FIGS. 6A-6B show the change in NRS in subjects of 18-80 years old following treatment with 1 mg or 5 mg of serlopitant (A) and percentage change in NRS 4-Point Responder Rate (B), respectively, in comparison to the placebo group. N is the number of tested subjects.

### DETAILED DESCRIPTION OF THE DISCLOSURE

While various embodiments of the present disclosure are described herein, it will be obvious to those skilled in the art that such embodiments are provided by way of example only. Numerous modifications and changes to, and variations and substitutions of, the embodiments described herein will be apparent to those skilled in the art without departing from the disclosure. It is understood that various alternatives to the embodiments described herein can be employed in practicing the disclosure. It is also understood that every embodiment of the disclosure can optionally be combined with any one or more of the other embodiments described herein which are consistent with that embodiment.

Where elements are presented in list format (e.g., in a Markush group), it is understood that each possible subgroup of the elements is also disclosed, and any one or more elements can be removed from the list or group.

It is also understood that, unless clearly indicated to the contrary, in any method described or claimed herein that includes more than one act, the order of the acts of the method is not necessarily limited to the order in which the acts of the method are recited, but the disclosure encompasses embodiments in which the order is so limited.

It is further understood that, in general, where an embodiment in the description or the claims is referred to as comprising one or more features, the disclosure also encompasses embodiments that consist of, or consist essentially of, such feature(s).

It is also understood that any embodiment of the disclosure, e.g., any embodiment found within the prior art, can be explicitly excluded from the claims, regardless of whether or not the specific exclusion is recited in the specification.

It is further understood that the present disclosure encompasses analogs, derivatives, prodrugs, metabolites, salts, solvates, hydrates, clathrates, polymorphs and stereoisomers of all of the compounds/substances disclosed herein, as appropriate. The specific recitation of “analogs”, “derivatives”. “prodrugs”, “metabolites”, “salts”, “solvates”, “hydrates”, “clathrates”, “polymorphs” or “stereoisomers” with respect to a compound/substance or a group of compounds/substances in certain instances of the disclosure shall not be interpreted as an intended omission of any of these forms in other instances of the disclosure where the compound/substance or the group of compounds/substances is mentioned without recitation of any of these forms, unless stated otherwise or the context clearly indicates otherwise.

Headings are included herein for reference and to aid in locating certain sections. Headings are not intended to limit the scope of the embodiments and concepts described in the sections under those headings, and those embodiments and concepts may have applicability in other sections throughout the entire disclosure.

All patent literature and all non-patent literature cited herein are incorporated herein by reference in their entirety to the same extent as if each patent literature or non-patent literature were specifically and individually indicated to be incorporated herein by reference in its entirety.

### Definitions

Unless defined otherwise or clearly indicated otherwise by their use herein, all technical and scientific terms used herein have the same meaning as commonly understood by one of ordinary skill in the art to which this application belongs.

As used in the specification and the appended claims, the indefinite articles “a” and “an” and the definite article “the” can include plural referents as well as singular referents unless specifically stated otherwise or the context clearly dictates otherwise.

The abbreviation “aka” denotes “also known as”.

The term “about” or “approximately” means an acceptable error for a particular value as determined by one of ordinary skill in the art, which depends in part on how the value is measured or determined. In certain embodiments, the term “about” or “approximately” means within one standard deviation. In some embodiments, when no particular margin of error (e.g., a standard deviation to a mean value given in a chart or table of data) is recited, the term “about” or “approximately” means that range which would encompass the recited value and the range which would be included by rounding up or down to the recited value as well, taking into account significant figures. In certain embodiments, the term “about” or “approximately” means within ±20%, 15%, 10% or 5% of the specified value. Whenever the term “about” or “approximately” precedes the first numerical value in a series of two or more numerical values or in a series of two or more ranges of numerical values, the term “about” or “approximately” applies to each one of the numerical values in that series of numerical values or in that series of ranges of numerical values.

Whenever the term “at least” or “greater than” precedes the first numerical value in a series of two or more numerical values, the term “at least” or “greater than” applies to each one of the numerical values in that series of numerical values.

Whenever the term “no more than” or “less than” precedes the first numerical value in a series of two or more numerical values, the term “no more than” or “less than” applies to each one of the numerical values in that series of numerical values.

The term “antagonists” includes neutral antagonists and inverse agonists.

The term “pharmaceutically acceptable” refers to a substance (e.g., an active ingredient or an excipient) that is suitable for use in contact with the tissues and organs of a subject without excessive irritation, allergic response, immunogenicity and toxicity, is commensurate with a reasonable benefit/risk ratio, and is effective for its intended use. A “pharmaceutically acceptable” carrier or excipient of a pharmaceutical composition is also compatible with the other ingredients of the composition.

The term “therapeutically effective amount” refers to an amount of a substance that, when administered to a subject, is sufficient to prevent, reduce the risk of developing, delay the onset of, slow the progression of, or cause regression of the medical condition being treated, or to alleviate to some extent the medical condition or one or more symptoms or complications of that condition. The term “therapeutically effective amount” also refers to an amount of a substance that is sufficient to elicit the biological or medical response of a cell, tissue, organ, system, animal or human which is sought by a researcher, veterinarian, medical doctor or clinician.

The terms “treat”, “treating” and “treatment” include alleviating, ameliorating or abrogating a medical condition or one or more symptoms or complications associated with the condition, and alleviating, ameliorating or eradicating one or more causes of the condition. Reference to “treatment” of a medical condition includes preventing (precluding), reducing the risk of developing, delaying the onset of, slowing the progression of, and causing regression of the condition or one or more symptoms or complications associated with the condition.

The term “medical conditions” (or “conditions” for short) includes disorders and diseases. The terms “disorders” and “diseases” are used interchangeably herein.

The term “subject” refers to an animal, including a mammal, such as a primate (e.g., a human, a chimpanzee or a monkey), a rodent (e.g., a rat, a mouse, a gerbil, a hamster or a guinea pig), a lagomorph (e.g., a rabbit), a swine (e.g., a pig), an equine (e.g., a horse), a canine (e.g., a dog) or a feline (e.g., a cat). The terms “subject” and “patient” are used interchangeably herein in reference, e.g., to a mammalian subject, such as a human subject.

### The Role of Substance P and Neurokinin-1 in Pruritus

An important pruritus pathway is mediated by the neuropeptide substance P. Substance P is the most potent tachykinin and binds most strongly to neurokinin-1 (NK-1, also called tachykinin receptor 1 or substance P receptor) among the three tachykinin receptors NK-1, NK-2 and NK-3. NK-1 is expressed in the PNS, including on keratinocytes and immune cells (e.g, mast cells) in the skin, and the CNS, including the dorsal root ganglia (DRG) of spinal nerves, the spinal dorsal horn and the brain. Substance P activates NK-1 in the PNS and the CNS, and the substance P/NK-1 interaction is an important mediator of the induction and maintenance of pruritus. Substance P and NK-1 receptors are overexpressed in pruritic human skin, and the skin of patients with chronic pruritus has a significantly greater density of substance P sensory nerve fibers compared to normal skin. Furthermore, injection of substance P into human skin causes symptoms of neurogenic inflammation such as erythema, edema and intense itch. Moreover, NK-1 receptors in the dorsal root ganglia of rats mediate scratching behavior.

The pruritogenic effect of substance P is intertwined with its pro-inflammatory effects. Activated pruriceptive neurons, including unmyelinated C nerve fibers, release neuropeptides such as calcitonin gene-related peptide (CGRP) and substance P into the surrounding tissues. Substance P binding to NK-1 on keratinocytes and fibroblasts in the skin stimulates the secretion of inflammatory factors such as histamine, serotonin, interferon-γ, interleukin-1β (IL-1β), IL-6, IL-8 and nerve growth factor (NGF). Moreover, substance P binding to NK-1 or MrgprX2 on mast cells in the skin leads to degranulation and secretion of inflammatory factors such as histamine, serotonin, leukotriene B4, prostaglandins D2 and E2, IL-2, IL-6, IL-8, IL-31, tumor necrosis factor-alpha (TNF-α), NGF, vascular endothelial growth factor (VEGF) and proteases (e.g., tryptase). The pro-inflammatory factors released from keratinocytes, fibroblasts and mast cells take part in the pathogenesis of pruritus, including by stimulating vasodilation and neurogenic inflammation, whose symptoms include erythema, edema and burning itch. Substance P binding to NK-1 on blood vessels also leads to vasodilation and neurogenic inflammation. Certain pruritogens including histamine, neuropeptides (e.g., substance P, gastrin-releasing peptide [GRP], neurotensin, somatostatin and vasoactive intestinal peptide [VIP]), interleukins (e.g., IL-31, whose receptor is expressed on cutaneous C nerve fibers, keratinocytes and DRG neurons), and proteases (e.g., tryptase) provoke itch directly by binding to pruriceptors or indirectly by inducing release of histamine or other pruritogens. For example, histamine induces itch by stimulating the histamine H₁ and H₄ receptors on the endings of mechano-insensitive C nerve fibers in the skin (histamine also activates the histamine H₄ receptor on inflammatory cells including mast cells and T-lymphocytes [e.g., Th2 cells], thereby intensifying the pruritic signal), proteases (e.g., tryptase) activate the pruriceptor protease-activated receptor 2 (PAR2) on the endings of mechano-sensitive C nerve fibers in the skin, and substance P and GRP induce itch by promoting release of various pruritogens such as histamine and proteases (e.g., tryptase) from, e.g., mast cells in the skin. Binding of pruritogens to their respective receptors on unmyelinated C fibers or thinly myelinated Δδ fibers in the skin activates the neurons and results in the opening of TRPV1 or TRPA1 ion channels, which leads to neuronal depolarization, action potential (AP) firing and transmission of itch signals to the CNS.

Primary afferents within the skin transmit sensory information from their nerve endings to their cell bodies in the DRG and trigeminal ganglia. The afferent nerve fibers within the skin are mostly slow-conducting, small-diameter, unmyelinated C fibers (conduction from 0.5 m/sec to 2 m/sec); medium-diameter, thinly myelinated Δδ fibers; and some fast-conducting, larger-diameter, myelinated Aβ fibers (conduction from 4 m/sec to 70 m/sec). The A fibers transmit tactile sensitivity, temperature and noxious sensations and have a role in the perception of itch. About 80% of the C fibers are polymodal as they can respond to thermal, mechanical and chemical stimuli. The remaining 20% of the C fibers are not responsive to mechanical stimuli, and only about 5% of these C fibers are specific for itch. Free C-fiber nerve endings are located in the dermis and epidermis, and the thin axons of C fibers extend from the epidermis to the nerve cell body. Interactions of the axons with pruritogens, epithelial cells and immune cells can trigger an itch sensation. Itch is detected mainly by C fibers and Aδ fibers. Under inflammatory or/and chronic itch conditions, itch receptors (pruriceptors) are sensitized and become receptive to mechanical stimuli, and the non-itch, mechanoreceptor Aβ fibers within the skin also become sensitized and can transmit the itch signal (alloknesis), his leads to an increase in stimuli reception and broadcasting. When the Aδ fibers or C fibers become sensitized, they have a lower itch-evoking threshold or/and provide a stronger itch signal (hyperknesis), and stimuli are no longer needed to continue the itch cycle (spontaneous itch).

The central branches of itch-sensitive primary sensory neurons, mainly C fibers and Δδ fibers, terminate in superficial laminas of the spinal or medullary dorsal horn to activate second-order sensory neurons. Pruriceptive primary afferents convey the itch signal by releasing specific neurotransmitters onto postsynaptic neurons in the superficial dorsal horn of the spinal cord and the trigeminal subnucleus caudalis (Vc), where the itch signal as well as descending synaptic inputs from the brain are processed by local excitatory and inhibitory neurons. The itch information is then transmitted via ascending axons to the contralateral ventrobasal thalamus (spinothalamic tract) and the lateral parabrachial (PB) nucleus bilaterally (spinoparabrachial tract). The ventral posterior nucleus in the thalamus relays somatosensory information such as itch, and the PB nucleus is connected to the amygdala, the hypothalamus and the insular cortex. Most pruriceptive neurons in the superficial dorsal horn of the spinal cord and the Vc are interneurons, while a minority of them are projection neurons that innervate the thalamus or the PB nucleus. Without intending to be bound by theory, itch-signal transmission is believed to involve the release of brain natriuretic peptide (BNP) from the central terminals of pruriceptive C and Aδ fibers, which activates atrial natriuretic peptide receptor (NprA)-expressing interneurons, which in turn release gastrin-releasing peptide (GRP), which activates GRPR-expressing interneurons, which in turn release substance P, which activates NK1-expressing projection neurons that transmit itch information to the brain. Alternatively, GRP, or both GRP and BNP, may be released from the central terminals of pruriceptive primary afferents. The brain perceives itch and activates the motor system to initiate scratching, which relieves itch. Scratching stimulates inhibitory spinal interneurons (e.g., those expressing the transcription factor BhlhB5) to release the κ-opioid receptor-activating dynorphins and the inhibitory neurotransmitters γ-aminobutyric acid (GABA) and glycine, all of which inhibit itch-signaling neurons. However, rebound of AP firing by itch-signaling central neurons occurs after scratching ceases.

Scratching provoked by itch damages the skin, consequently maintaining and reinforcing the inflammatory processes that promote nerve fiber activation and induce further pruritus. During inflammation, keratinocytes, fibroblasts, and certain immune cells (mast cells, macrophages, cosinophils and neutrophils) release pruritogens such as histamine, tryptase, neurotrophins, leukotrienes, interleukins and TNF-α. Scratching results in local proliferation of skin nerves, mast cell degranulation and increase in the levels of neuropeptides including substance P (e.g., skin scratching leads to upregulation of NK-1 on epidermal keratinocytes and release of substance P from sensory C fibers), which leads to increased secretion of cytokines and other pro-inflammatory mediators and stimulation of keratinocytes, fibroblasts and mast cells, thereby creating an itch/scratch cycle.

Without intending to be bound by theory, chronic itch is believed to result from or/and to contribute to increased sensitivity of peripheral or/and central itch-signaling neurons that hence have a lower itch-evoking threshold or/and provide a stronger itch signal to the CNS, leading to spontaneously occurring itch, alloknesis and hyperknesis. Inflammatory mediators such as bradykinin, prostaglandins, interleukins and neurotrophins (e.g., NGF) sensitize peripheral itch sensory neurons, which is mediated in part by, e.g., TRPA1, TRPV1, Nav1.7, MrgprA3, PAR2 and toll-like receptors (e.g., TLR3). Peripheral sensitization increases the excitability and activity of primary itch-sensing neurons through hyperinnervation or/and lowering of the threshold of receptors on peripheral nerves to stimuli such that sensitized peripheral nerves react to stimuli that normally induce no, or a much weaker, itch response. Elevated levels of neurotrophins such as NGF also promote sprouting and elongation of itch-sensitive nerve fibers into the epidermis and their survival. Peripheral sensitization triggers central sensitization, which is mediated by increased excitatory synaptic transmission and reduced inhibitory synaptic transmission. Peripheral and central sensitization causes glial activation (e.g., astrogliosis in the spinal dorsal horn) and neuroinflammation in the CNS, which result in the production of cytokines (e.g., TNF-α, IL-1β and IL-6), chemokines {e.g., (C—C motif) ligand 2 (CCL2) and (C—X—C motif) ligand 1 (CXCL1)} and neurotrophins (e.g., brain-derived neurotrophic factor [BDNF]) that maintain central sensitization, leading to chronic itch. In central sensitization, ongoing activation of, e.g., unmyelinated C fibers in patients with chronic pruritus lowers the threshold of second-order neurons in the spinal cord such that sensitized second-order neurons become activated by itch signals that normally are not sufficiently intense, or continuously activates the second-order neurons such that they provide an ongoing or/and stronger itch signal to the brain. In addition to peripheral and central sensitization, impaired inhibition of itch signaling in the spinal or/and medullary dorsal horn can perpetuate pruritus.

As mentioned above, substance P is a key neuropeptide transmitter that is released from activated excitatory spinal interneurons and activates NK1-expressing spinal dorsal horn neurons. Most spinal dorsal horn neurons with ascending axonal projections to the thalamus and the PB nucleus express NK-1, and such NK-expressing projection neurons transmit itch information to the brain. NK1-expressing spinal dorsal horn neurons are major contributors to chronic itch (ongoing itch), spontaneous itch, alloknesis (itch induced by a normally non-itchy stimulus, such as light touch), and hyperknesis (enhanced itch induced by a normally itchy stimulus). Therefore. NK1-expressing spinal dorsal horn neurons play an important role in chronic itch and the development and maintenance of itch sensitization regardless of the pruritogenic stimuli, and inhibition of such neurons using an NK-1 antagonist can curtail chronic itch and itch sensitization regardless of the pruritogenic stimuli.

Peripheral and central mechanisms of itch and the role of substance P and NK-1 in itch are discussed in, e.g., T. Akiyama et al., Pain, 156:1240-1246 (2015); E. Carstens and T. Akiyama, Curr. Probl. Dermatol., 50:11-17 (2016); J. S. Lee et al., BMB Rep., 49:474-487 (2016); and T. Lotts and S. Stnder, J. Dtsch. Dermatol. Ges., 12:557-559 (2014).

### Treatment of Pruritus Using Neurokinin-1 Antagonists

The present disclosure provides for the use of a neurokinin-1 (NK-1) antagonist in treating pruritus in subjects at least 18 years of age (e.g., at least 40 years of age), having the disease for one year or longer, having no inflammatory skin disease, and/or having idiopathic pruritus. In another aspect, the present disclosure provides for the use of a neurokinin-1 (NK-1) antagonist in treating pruritus in subjects 18 years of age (e.g., at least at least 40 years of age), having the disease for one year or longer, having no inflammatory skin disease, and/or having pruritus of unknown origin. In some embodiments, the subject has no inflammatory skin disease at the time of administration of the NK-1 antagonist. In some embodiments, the subject has no history of inflammatory skin disease. In some embodiments, the subject has no inflammatory skin disease at the time of administration of the NK-1 antagonist but has a history of inflammatory skin disease. In some embodiments, the subject has no inflammatory skin disease at the time of administration of the NK-1 antagonist and has no history of inflammatory skin disease.

In another aspect pruritus is associated with aging (e.g., elderly pruritus), as manifested, for example, by increased skin dryness, neuropathic changes, immunosenescence, or a combination of the foregoing. In some embodiments, the age-associated pruritus is of unknown origin. In some embodiments, the age-related pruritus is of dermal or neuropathic origin. In some embodiments, the age-associated pruritus is idiopathic. In some embodiments, the age-associated pruritus is chronic pruritus. In some embodiments, the duration of the age-associated pruritus is at least 6 months, 1 year, at least 2 years, at least 3 years, at least 4 years, or at least 5 years. In some embodiments, the subject with age-associated pruritus is at least 30 years old, at least 35 years old, at least 40 years old, at least 45 years old, at least 50 years old, at least 55 years old, at least 60 years old, at least 65 years old, at least 70 years old, at least 75 years old, at least 80 years old, at least 85 years old, or at least 90 years old. In some embodiments, the subject with age-associated pruritus is about 30-80 years old, about 30-40 years old, about 40-50 years old, about 50-60 years old, about 60-70 years old, about 70-80 years old, about 80-90 years old, or about 50-80 years old.

In some embodiments, the subject is at least 18 years of age, at least 20 years of age, at least 25 years of age, at least 30 years of age, at least 35 years of age, at least 40 years of age, at least 45 years of age, at least 50 years of age, at least 55 years of age, at least 60 years of age, at least 65 years of age, at least 70 years of age, at least 75 years of age, at least 80 years of age, at least 85 years of age, or at least 90 years of age. In some embodiments, the subject is about 18-20 years of age, about 20-25 years of age, about 25-30 years of age, about 30-35 years of age, about 35-40 years of age, about 40-45 years of age, about 45-50 years of age, about 50-55 years of age, about 55-60 year of age, about 60-65 years of age, about 65-70 years of age, about 70-75 years of age, about 75-80 years of age, about 80-85 years of age, about 85-90 years of age, about 40-50 years of age, about 50-60 years of age, about 60-70 years of age, about 70-80 years of age, about 80-90 years of age, or about 90-100 years of age.

In some embodiments, the duration of the pruritus is about one year or longer, about 1.5 years or longer, about 2 years or longer, about 2.5 years or longer, about 3 years or longer, about 3.5 years or longer, about 4 years or longer, about 4.5 years or longer, about 5 years or longer, about 6 years or longer, about 7 years or longer, about 8 years or longer, about 9 years or longer, about 10 years or longer, about 12 years or longer, about 14 years or longer, about 15 years or longer, about 16 year or longer, about 18 years or longer, or about 20 years or longer. In some embodiments, the duration of the pruritus is about 1-1.5 years, about 1.5-2 years, about 2-2.5 years, about 2.5-3 years, about 3-3.5 years, about 3.5-4 years, about 4-4.5 years, about 4.5-5 years, about 5-5.5 years, about 5.5-6 years, about 6-6.5 years, about 6.5-7 years, about 7-7.5 years, about 7.5-8 years, about 8-8.5 years, about 8.5-9 years, about 9-10 years, about 10-12 years, about 12-15 years, about 15-17 years, about 17-20 years, about 1-3 years, about 3-5 years, about 5-7 years, about 7-10 years, about 10-15 years, or about 15-20 years.

In some embodiments, the subject has no inflammatory skin disease. In some embodiments, the pruritus is idiopathic. In some embodiments, the pruritus is of unknown origin. In some embodiments, the pruritus is chronic. In some embodiments, the pruritus is chronic pruritus of unknown origin. In some embodiments, the subject has no inflammatory skin disease and the pruritus is idiopathic. In some embodiments, the subject has no inflammatory skin disease and the pruritus is of unknown origin. In some embodiments, the subject has no inflammatory skin disease at the time of administration of the NK-1 antagonist. In some embodiments, the subject has no history of inflammatory skin disease. In some embodiments, the subject has no inflammatory skin disease at the time of administration of the NK-1 antagonist but has a history of inflammatory skin disease. In some embodiments, the subject has no inflammatory skin disease at the time of administration of the NK-1 antagonist and has no history of inflammatory skin disease. In some embodiments, the subject has no inflammatory skin disease and the subject is at least 18 years of age, at least 20 years of age, at least 25 years of age, at least 30 years of age, at least 35 years of age, at least 40 years of age, at least 45 years of age, at least 50 years of age, at least 55 years of age, at least 60 years of age, at least 65 years of age, at least 70 years of age, at least 75 years of age, at least 80 years of age, at least 85 years of age, or at least 90 years of age. In some embodiments, the subject has no inflammatory skin disease and the subject is about 18-20 years of age, about 20-25 years of age, about 25-30 years of age, about 30-35 years of age, about 35-40 years of age, about 40-45 years of age, about 45-50 years of age, about 50-55 years of age, about 55-60 year of age, about 60-65 years of age, about 65-70 years of age, about 70-75 years of age, about 75-80 years of age, about 80-85 years of age, about 85-90 years of age, about 40-50 years of age, about 50-60 years of age, about 60-70 years of age, about 70-80 years of age, about 80-90 years of age, or about 90-100 years of age. In some embodiments, the subject has no inflammatory skin disease and the duration of the pruritus is about one year or longer, about 1.5 years or longer, about 2 years or longer, about 2.5 years or longer, about 3 years or longer, about 3.5 years or longer, about 4 years or longer, about 4.5 years or longer, about 5 years or longer, about 6 years or longer, about 7 years or longer, about 8 years or longer, about 9 years or longer, about 10 years or longer, about 12 years or longer, about 14 years or longer, about 15 years or longer, about 16 years or longer, about 18 years or longer, or about 20 years or longer. In some embodiments, the subject has no inflammatory skin disease and the duration of the pruritus is about 1-1.5 years, about 1.5-2 years, about 2-2.5 years, about 2.5-3 years, about 3-3.5 years, about 3.5-4 years, about 4-4.5 years, about 4.5-5 years, about 5-5.5 years, about 5.5-6 years, about 6-6.5 years, about 6.5-7 years, about 7-7.5 years, about 7.5-8 years, about 8-8.5 years, about 8.5-9 years, about 9-10 years, about 10-12 years, about 12-15 years, about 15-17 years, about 17-20 years, about 1-3 years, about 3-5 years, about 5-7 years, about 7-10 years, about 10-15 years, or about 15-20 years.

In some embodiments, the pruritus is idiopathic and the subject is at least 18 years of age, at least 20 years of age, at least 25 years of age, at least 30 years of age, at least 35 years of age, at least 40 years of age, at least 45 years of age, at least 50 years of age, at least 55 years of age, at least 60 years of age, at least 65 years of age, at least 70 years of age, at least 75 years of age, at least 80 years of age, at least 85 years of age, or at least 90 years of age. In some embodiments, the pruritus is idiopathic and the subject is about 18-20 years of age, about 20-25 years of age, about 25-30 years of age, about 30-35 years of age, about 40-45 years of age, about 45-50 years of age, about 50-55 years of age, about 55-60 year of age, about 60-65 years of age, about 65-70 years of age, about 70-75 years of age, about 75-80 years of age, about 80-85 years of age, about 85-90 years of age, about 40-50 years of age, about 50-60 years of age, about 60-70 years of age, about 70-80 years of age, about 80-90 years of age, or about 90-100 years of age. In some embodiments, the pruritus is idiopathic and the duration of the pruritus is about one year or longer, about 1.5 years or longer, about 2 years or longer, about 2.5 years or longer, about 3 years or longer, about 3.5 years or longer, about 4 years or longer, about 4.5 years or longer, about 5 years or longer, about 6 years or longer, about 7 years or longer, about 8 years or longer, about 9 years or longer, about 10 years or longer, about 12 years or longer, about 14 years or longer, about 15 years or longer, about 16 years or longer, about 18 years or longer, or about 20 years or longer. In some embodiments, the pruritus is idiopathic and the duration of the pruritus is about 1-1.5 years, about 1.5-2 years, about 2-2.5 years, about 2.5-3 years, about 3-3.5 years, about 3.5-4 years, about 4-4.5 years, about 4.5-5 years, about 5-5.5 years, about 5.5-6 years, about 6-6.5 years, about 6.5-7 years, about 7-7.5 years, about 7.5-8 years, about 8-8.5 years, about 8.5-9 years, about 9-10 years, about 10-12 years, about 12-15 years, about 15-17 years, about 17-20 years, about 1-3 years, about 3-5 years, about 5-7 years, about 7-10 years, about 10-15 years, or about 15-20 years.

In some embodiments, the pruritus is of unknown origin and the subject is at least at least 18 years of age, at least 20 years of age, at least 25 years of age, at least 30 years of age, at least 35 years of age, 40 years of age, at least 45 years of age, at least 50 years of age, at least 55 years of age, at least 60 years of age, at least 65 years of age, at least 70 years of age, at least 75 years of age, at least 80 years of age, at least 85 years of age, or at least 90 years of age. In some embodiments, the pruritus is of unknown origin and the subject is about 18-20 years of age, about 20-25 years of age, about 25-30 years of age, about 30-35 years of age, about 35-40 years of age, about 40-45 years of age, about 45-50 years of age, about 50-55 years of age, about 55-60 year of age, about 60-65 years of age, about 65-70 years of age, about 70-75 years of age, about 75-80 years of age, about 80-85 years of age, about 85-90 years of age, about 40-50 years of age, about 50-60 years of age, about 60-70 years of age, about 70-80 years of age, about 80-90 years of age, or about 90-100 years of age. In some embodiments, the pruritus is of unknown origin and the duration of the pruritus is about one year or longer, about 1.5 years or longer, about 2 years or longer, about 2.5 years or longer, about 3 years or longer, about 3.5 years or longer, about 4 years or longer, about 4.5 years or longer, about 5 years or longer, about 6 years or longer, about 7 years or longer, about 8 years or longer, about 9 years or longer, about 10 years or longer, about 12 years or longer, about 14 years or longer, about 15 years or longer, about 16 years or longer, about 18 years or longer, or about 20 years or longer. In some embodiments, the pruritus is of unknown origin and the duration of the pruritus is about 1-1.5 years, about 1.5-2 years, about 2-2.5 years, about 2.5-3 years, about 3-3.5 years, about 3.5-4 years, about 4-4.5 years, about 4.5-5 years, about 5-5.5 years, about 5.5-6 years, about 6-6.5 years, about 6.5-7 years, about 7-7.5 years, about 7.5-8 years, about 8-8.5 years, about 8.5-9 years, about 9-10 years, about 10-12 years, about 12-15 years, about 15-17 years, about 17-20 years, about 1-3 years, about 3-5 years, about 5-7 years, about 7-10 years, about 10-15 years, or about 15-20 years.

In some embodiments, the subject has no inflammatory skin disease, the pruritus is idiopathic, and the subject is at least 18 years of age, at least 20 years of age, at least 25 years of age, at least 30 years of age, at least 35 years of age, at least 40 years of age, at least 45 years of age, at least 50 years of age, at least 55 years of age, at least 60 years of age, at least 65 years of age, at least 70 years of age, at least 75 years of age, at least 80 years of age, at least 85 years of age, or at least 90 years of age. In some embodiments, the subject has no inflammatory skin disease, the pruritus is idiopathic, and the subject is about 18-20 years of age, about 20-25 years of age, about 25-30 years of age, about 30-35 years of age, about 40-45 years of age, about 45-50 years of age, about 50-55 years of age, about 55-60 year of age, about 60-65 years of age, about 65-70 years of age, about 70-75 years of age, about 75-80 years of age, about 80-85 years of age, about 85-90 years of age, about 40-50 years of age, about 50-60 years of age, about 60-70 years of age, about 70-80 years of age, about 80-90 years of age, or about 90-100 years of age. In some embodiments, the subject has no inflammatory skin disease, the pruritus is idiopathic, and the duration of the pruritus is about one year or longer, about 1.5 years or longer, about 2 years or longer, about 2.5 years or longer, about 3 years or longer, about 3.5 years or longer, about 4 years or longer, about 4.5 years or longer, about 5 years or longer, about 6 years or longer, about 7 years or longer, about 8 years or longer, about 9 years or longer, about 10 years or longer, about 12 years or longer, about 14 years or longer, about 15 years or longer, about 16 years or longer, about 18 years or longer, or about 20 years or longer. In some embodiments, the subject has no inflammatory skin disease, the pruritus is idiopathic, and the duration of the pruritus is about 1-1.5 years, about 1.5-2 years, about 2-2.5 years, about 2.5-3 years, about 3-3.5 years, about 3.5-4 years, about 4-4.5 years, about 4.5-5 years, about 5-5.5 years, about 5.5-6 years, about 6-6.5 years, about 6.5-7 years, about 7-7.5 years, about 7.5-8 years, about 8-8.5 years, about 8.5-9 years, about 9-10 years, about 10-12 years, about 12-15 years, about 15-17 years, about 17-20 years, about 1-3 years, about 3-5 years, about 5-7 years, about 7-10 years, about 10-15 years, or about 15-20 years. In some embodiments, the subject has no inflammatory skin disease at the time of administration of the NK-1 antagonist. In some embodiments, the subject has no history of inflammatory skin disease. In some embodiments, the subject has no inflammatory skin disease at the time of administration of the NK-1 antagonist but has a history of inflammatory skin disease. In some embodiments, the subject has no inflammatory skin disease at the time of administration of the NK-1 antagonist and has no history of inflammatory skin disease.

In some embodiments, the subject has no inflammatory skin disease, the pruritus is of unknown origin, and the subject is at least 18 years of age, at least 20 years of age, at least 25 years of age, at least 30 years of age, at least 35 years of age, at least 40 years of age, at least 45 years of age, at least 50 years of age, at least 55 years of age, at least 60 years of age, at least 65 years of age, at least 70 years of age, at least 75 years of age, at least 80 years of age, at least 85 years of age, or at least 90 years of age. In some embodiments, the subject has no inflammatory skin disease, the pruritus is of unknown origin, and the subject is about 18-20 years of age, about 20-25 years of age, about 25-30 years of age, about 30-35 years of age, about 35-40 years of age, about 40-45 years of age, about 45-50 years of age, about 50-55 years of age, about 55-60 year of age, about 60-65 years of age, about 65-70 years of age, about 70-75 years of age, about 75-80 years of age, about 80-85 years of age, about 85-90 years of age, about 40-50 years of age, about 50-60 years of age, about 60-70 years of age, about 70-80 years of age, about 80-90 years of age, or about 90-100 years of age. In some embodiments, the subject has no inflammatory skin disease, the pruritus is of unknown origin, and the duration of the pruritus is about one year or longer, about 1.5 years or longer, about 2 years or longer, about 2.5 years or longer, about 3 years or longer, about 3.5 years or longer, about 4 years or longer, about 4.5 years or longer, about 5 years or longer, about 6 years or longer, about 7 years or longer, about 8 years or longer, about 9 years or longer, about 10 years or longer, about 12 years or longer, about 14 years or longer, about 15 years or longer, about 16 years or longer, about 18 years or longer, or about 20 years or longer. In some embodiments, the subject has no inflammatory skin disease, the pruritus is of unknown origin, and the duration of the pruritus is about 1-1.5 years, about 1.5-2 years, about 2-2.5 years, about 2.5-3 years, about 3-3.5 years, about 3.5-4 years, about 4-4.5 years, about 4.5-5 years, about 5-5.5 years, about 5.5-6 years, about 6-6.5 years, about 6.5-7 years, about 7-7.5 years, about 7.5-8 years, about 8-8.5 years, about 8.5-9 years, about 9-10 years, about 10-12 years, about 12-15 years, about 15-17 years, about 17-20 years, about 1-3 years, about 3-5 years, about 5-7 years, about 7-10 years, about 10-15 years, or about 15-20 years. In some embodiments, the subject has no inflammatory skin disease at the time of administration of the NK-1 antagonist. In some embodiments, the subject has no history of inflammatory skin disease. In some embodiments, the subject has no inflammatory skin disease at the time of administration of the NK-1 antagonist but has a history of inflammatory skin disease. In some embodiments, the subject has no inflammatory skin disease at the time of administration of the NK-1 antagonist and has no history of inflammatory skin disease.

In some embodiments, the subject has no inflammatory skin disease, the pruritus is idiopathic, the subject is at least 40 years of age, and the duration of pruritus is about one year or longer. In some embodiments, the subject has no inflammatory skin disease, the pruritus is of unknown origin, the subject is at least 40 years of age, and the duration of pruritus is about one year or longer. In some embodiments, the subject has no inflammatory skin disease, the pruritus is idiopathic, the subject is at least 18 years of age, at least 20 years of age, at least 25 years of age, at least 30 years of age, at least 35 years of age, at least 40 years of age, at least 45 years of age, at least 50 years of age, at least 55 years of age, at least 60 years of age, at least 65 years of age, at least 70 years of age, at least 75 years of age, at least 80 years of age, at least 85 years of age, or at least 90 years of age, and the duration of pruritus is about one year or longer, about 1.5 years or longer, about 2 years or longer, about 2.5 years or longer, about 3 years or longer, about 3.5 years or longer, about 4 years or longer, about 4.5 years or longer, about 5 years or longer, about 6 years or longer, about 7 years or longer, about 8 years or longer, about 9 years or longer, about 10 years or longer, about 12 years or longer, about 14 years or longer, about 15 years or longer, about 16 years or longer, about 18 years or longer, or about 20 years or longer. In some embodiments, the subject has no inflammatory skin disease, the pruritus is of unknown origin, the subject is at least 18 years of age, at least 20 years of age, at least 25 years of age, at least 30 years of age, at least 35 years of age, at least 40 years of age, at least 45 years of age, at least 50 years of age, at least 55 years of age, at least 60 years of age, at least 65 years of age, at least 70 years of age, at least 75 years of age, at least 80 years of age, at least 85 years of age, or at least 90 years of age, and the duration of pruritus is about one year or longer, about 1.5 years or longer, about 2 years or longer, about 2.5 years or longer, about 3 years or longer, about 3.5 years or longer, about 4 years or longer, about 4.5 years or longer, about 5 years or longer, about 6 years or longer, about 7 years or longer, about 8 years or longer, about 9 years or longer, about 10 years or longer, about 12 years or longer, about 14 years or longer, about 15 years or longer, about 16 years or longer, about 18 years or longer, or about 20 years or longer. In some embodiments, the subject has no inflammatory skin disease, the pruritus is idiopathic, the subject is about 18-20 years of age, about 20-25 years of age, about 25-30 years of age, about 30-35 years of age, about 35-40 years of age, about 40-45 years of age, about 45-50 years of age, about 50-55 years of age, about 55-60 year of age, about 60-65 years of age, about 65-70 years of age, about 70-75 years of age, about 75-80 years of age, about 80-85 years of age, about 85-90 years of age, about 40-50 years of age, about 50-60 years of age, about 60-70 years of age, about 70-80 years of age, about 80-90 years of age, or about 90-100 years of age, and the duration of the pruritus is about one year or longer, about 1.5 years or longer, about 2 years or longer, about 2.5 years or longer, about 3 years or longer, about 3.5 years or longer, about 4 years or longer, about 4.5 years or longer, about 5 years or longer, about 6 years or longer, about 7 years or longer, about 8 years or longer, about 9 years or longer, about 10 years or longer, about 12 years or longer, about 14 years or longer, about 15 years or longer, about 16 years or longer, about 18 years or longer, or about 20 years or longer. In some embodiments, the subject has no inflammatory skin disease, the pruritus is of unknown origin, the subject is about 18-20 years of age, about 20-25 years of age, about 25-30 years of age, about 30-35 years of age, about 35-40 years of age, about 40-45 years of age, about 45-50 years of age, about 50-55 years of age, about 55-60 year of age, about 60-65 years of age, about 65-70 years of age, about 70-75 years of age, about 75-80 years of age, about 80-85 years of age, about 85-90 years of age, about 40-50 years of age, about 50-60 years of age, about 60-70 years of age, about 70-80 years of age, about 80-90 years of age, or about 90-100 years of age, and the duration of the pruritus is about one year or longer, about 1.5 years or longer, about 2 years or longer, about 2.5 years or longer, about 3 years or longer, about 3.5 years or longer, about 4 years or longer, about 4.5 years or longer, about 5 years or longer, about 6 years or longer, about 7 years or longer, about 8 years or longer, about 9 years or longer, about 10 years or longer, about 12 years or longer, about 14 years or longer, about 15 years or longer, about 16 years or longer, about 18 years or longer, or about 20 years or longer. In some embodiments, the subject has no inflammatory skin disease, the pruritus is idiopathic, the subject is at least 40 years of age, at least 45 years of age, at least 50 years of age, at least 55 years of age, at least 60 years of age, at least 65 years of age, at least 70 years of age, at least 75 years of age, at least 80 years of age, at least 85 years of age, or at least 90 years of age, and the duration of pruritus is about 1-1.5 years, about 1.5-2 years, about 2-2.5 years, about 2.5-3 years, about 3-3.5 years, about 3.5-4 years, about 4-4.5 years, about 4.5-5 years, about 5-5.5 years, about 5.5-6 years, about 6-6.5 years, about 6.5-7 years, about 7-7.5 years, about 7.5-8 years, about 8-8.5 years, about 8.5-9 years, about 9-10 years, about 10-12 years, about 12-15 years, about 15-17 years, about 17-20 years, about 1-3 years, about 3-5 years, about 5-7 years, about 7-10 years, about 10-15 years, or about 15-20 years. In some embodiments, the subject has no inflammatory skin disease, the pruritus is of unknown origin, the subject is at least 18 years of age, at least 20 years of age, at least 25 years of age, at least 30 years of age, at least 35 years of age, at least 40 years of age, at least 45 years of age, at least 50 years of age, at least 55 years of age, at least 60 years of age, at least 65 years of age, at least 70 years of age, at least 75 years of age, at least 80 years of age, at least 85 years of age, or at least 90 years of age, and the duration of pruritus is about 1-1.5 years, about 1.5-2 years, about 2-2.5 years, about 2.5-3 years, about 3-3.5 years, about 3.5-4 years, about 4-4.5 years, about 4.5-5 years, about 5-5.5 years, about 5.5-6 years, about 6-6.5 years, about 6.5-7 years, about 7-7.5 years, about 7.5-8 years, about 8-8.5 years, about 8.5-9 years, about 9-10 years, about 10-12 years, about 12-15 years, about 15-17 years, about 17-20 years, about 1-3 years, about 3-5 years, about 5-7 years, about 7-10 years, about 10-15 years, or about 15-20 years. In some embodiments, the subject has no inflammatory skin disease, the pruritus is idiopathic, the subject is about 18-20 years of age, about 20-25 years of age, about 25-30 years of age, about 30-35 years of age, about 35-40 years of age, about 40-45 years of age, about 45-50 years of age, about 50-55 years of age, about 55-60 year of age, about 60-65 years of age, about 65-70 years of age, about 70-75 years of age, about 75-80 years of age, about 80-85 years of age, about 85-90 years of age, about 40-50 years of age, about 50-60 years of age, about 60-70 years of age, about 70-80 years of age, about 80-90 years of age, or about 90-100 years of age, and the duration of the pruritus is about 1-1.5 years, about 1.5-2 years, about 2-2.5 years, about 2.5-3 years, about 3-3.5 years, about 3.5-4 years, about 4-4.5 years, about 4.5-5 years, about 5-5.5 years, about 5.5-6 years, about 6-6.5 years, about 6.5-7 years, about 7-7.5 years, about 7.5-8 years, about 8-8.5 years, about 8.5-9 years, about 9-10 years, about 10-12 years, about 12-15 years, about 15-17 years, about 17-20 years, about 1-3 years, about 3-5 years, about 5-7 years, about 7-10 years, about 10-15 years, or about 15-20 years. In some embodiments, the subject has no inflammatory skin disease, the pruritus is of unknown origin, the subject is about 18-20 years of age, about 20-25 years of age, about 25-30 years of age, about 30-35 years of age, about 35-40 years of age, about 40-45 years of age, about 45-50 years of age, about 50-55 years of age, about 55-60 year of age, about 60-65 years of age, about 65-70 years of age, about 70-75 years of age, about 75-80 years of age, about 80-85 years of age, about 85-90 years of age, about 40-50 years of age, about 50-60 years of age, about 60-70 years of age, about 70-80 years of age, about 80-90 years of age, or about 90-100 years of age, and the duration of the pruritus is about 1-1.5 years, about 1.5-2 years, about 2-2.5 years, about 2.5-3 years, about 3-3.5 years, about 3.5-4 years, about 4-4.5 years, about 4.5-5 years, about 5-5.5 years, about 5.5-6 years, about 6-6.5 years, about 6.5-7 years, about 7-7.5 years, about 7.5-8 years, about 8-8.5 years, about 8.5-9 years, about 9-10 years, about 10-12 years, about 12-15 years, about 15-17 years, about 17-20 years, about 1-3 years, about 3-5 years, about 5-7 years, about 7-10 years, about 10-15 years, or about 15-20 years. In some embodiments, the subject has no inflammatory skin disease at the time of administration of the NK-1 antagonist. In some embodiments, the subject has no history of inflammatory skin disease. In some embodiments, the subject has no inflammatory skin disease at the time of administration of the NK-1 antagonist but has a history of inflammatory skin disease. In some embodiments, the subject has no inflammatory skin disease at the time of administration of the NK-1 antagonist and has no history of inflammatory skin disease.

In some embodiments, the subject has no more than about 5%, 7.5%, 10%, 12.5%, 15%, 17.5%, 20%, 25%, or 30% body surface area coverage with psoriasis lesions. In some embodiments, the psoriasis lesions are active psoriasis lesions. In some embodiments, the subject has about 5-7.5%, 7.5-10%, 10-12.5%, 12.5-15%, 15-17.5%, 17.5-20%, 20-25%, 25-30%, 5-10%, 10-15%, 15-20%, 20-30%, or 5-15% body surface area coverage with psoriasis lesions. In some embodiments, the psoriasis lesions are active psoriasis lesions. In some embodiments, the subject has no more than 10% body surface area coverage with active psoriasis lesions.

In some embodiments, the pruritus is characterized by sensitization or hypersensitization of the CNS. In certain embodiments, the pruritus is characterized by sensitization or hypersensitization of dorsal root ganglion neurons, spinal dorsal horn neurons or spinal trigeminal nucleus (e.g., medullary dorsal horn) neurons, or any combination or all thereof. In further embodiments, the pruritus is characterized by sensitization or hypersensitization of the PNS. In certain embodiments, the pruritus is characterized by sensitization or hypersensitization of unmyelinated C fibers, thinly myelinated Δδ fibers or myelinated Δβ fibers, or any combination or all thereof.

In some embodiments, the pruritus is characterized by spontaneously occurring itch, alloknesis or hyperknesis, or any combination or all thereof. In further embodiments, the NK-1 antagonist inhibits spontaneously occurring itch, alloknesis or hyperknesis, or any combination or all thereof. In certain embodiments, the NK-1 antagonist reduces the frequency or/and the intensity of spontaneously occurring itch, alloknesis or hyperknesis, or any combination or all thereof, by at least about 20%, 30%, 40%, 50%, 60%, 70%, 800%, 90% or 95% (e.g., by at least about 30% or 50%), as measured by a visual analog scale (VAS) score or a numerical rating scale (NRS) score, for example. In some embodiments, the NK-1 antagonist statistically significantly reduces the frequency or/and the intensity of spontaneously occurring itch, alloknesis or hyperknesis, or any combination or all thereof. In certain embodiments, the NK-1 antagonist reduces the frequency or/and the intensity of spontaneously occurring itch, alloknesis or hyperknesis, or any combination or all thereof, at least about 20%, 30%, 40%, 50%, 100%, 150% or 200% (e.g., at least about 30% or 50%) more than placebo, as measured by, e.g., a VAS or NRS score and as a percentage of the effect of placebo.

Non-limiting examples of NK-1 antagonists include aprepitant (L-754030 or MK-(0)869), fosaprepitant (L-758298), befetupitant, burapitant (SSR-240600), casopitant (GW-679769), dapitant (RPR-100893), ezlopitant (CJ-11974), figopitant (BIIF-1149), lanepitant (LY-303870), maropitant (CJ-11972), netupitant, fosnetupitant, nolpitantium (SR-140333), orvepitant (GW-823296), rolapitant (SCH-619734), SCH-720881 (active metabolite of rolapitant), serlopitant (MK-(0)594 or VPD-737), tradipitant (VLY-686 or LY-686017), vestipitant (GW-597599), vofopitant (GR-205171), hydroxyphenyl propamidobenzoic acid, maltooligosaccharides (e.g., maltotetraose and maltopentaose), spantides (e.g., spantide I and II), AV-608, AV-818, AZD-2624, CGP-49823, CJ-17493, CP-96345, CP-99994, CP-122721, DNK-333 (NK1/NK2), FK-224 (NK1/NK2), FK-888, GR-82334, GR-203040, GR-205171, GSK-424887, HSP-117, KRP-103, L-703606, L-733060, L-736281, L-759274, L-760735, LY-686017, M516102, MDL-105212 (NK1/NK2), MK-0303 (L-00l182885), MK-8478 (L-001983867), NKP-608, PD-154075, R-116031, R-116301, RP-67580, S-41744, SCH-206272 (NK-1/NK-2/NK-3), SCH-388714, SCH-900978, SLV-317, T-2328, TA-5538, TAK-637, TKA-731, WIN-51708, ZD-4974, ZD-6021 (NK1/NK2), and analogs, derivatives, prodrugs, metabolites, salts and stereoisomers thereof. In some embodiments, the NK-1 antagonist is aprepitant, fosaprepitant, rolapitant, orvepitant, serlopitant, or tradipitant, or a pharmaceutically acceptable salt, solvate, hydrate, clathrate, polymorph, prodrug, metabolite or stereoisomer thereof.

In some embodiments, the NK-1 antagonist is a selective NK-1 antagonist. In certain embodiments, the NK-1 antagonist is serlopitant (described below) or a pharmaceutically acceptable salt, solvate, hydrate, clathrate, polymorph, prodrug, metabolite or stereoisomer thereof. In other embodiments, the NK-1 antagonist is not aprepitant or fosaprepitant.

The therapeutically effective amount and the frequency of administration of, and the length of treatment with, the NK-1 antagonist to treat chronic pruritus may depend on various factors, including the nature and the severity of the pruritus or the underlying medical condition, the potency of the NK-1 antagonist, the route of administration, the age, the body weight, the general health, the gender and the diet of the subject, and the response of the subject to the treatment, and can be determined by the treating physician. In some embodiments, the therapeutically effective amount of the NK-1 antagonist for treating chronic pruritus is about 0.25 or 1 to 200 mg, 0.5 or 1 to 150 mg, 0.5 or 1 to 100 mg, 0.5 or 1 to 50 mg, 0.5 or 1 to 10 mg, 10-20 mg, 20-30 mg, 30-40 mg, 40-50 mg, 50-100 mg, 100-150 mg or 150-200 mg (e.g., per day or per dose), or as deemed appropriate by the treating physician, which can be administered in a single dose or in divided doses. In certain embodiments, the therapeutically effective dose (e.g., per day or per dose) of the NK-1 antagonist for treating chronic pruritus is about 0.5 or 1 to 5 mg (e.g., about 0.5 mg, 1 mg, 2 mg, 3 mg, 4 mg or 5 mg), about 5-10 mg (e.g., about 5 mg, 6 mg, 7 mg, 8 mg, 9 mg or 10 mg), about 10-20 mg (e.g., about 10 mg, 15 mg or 20 mg), about 20-30 mg (e.g., about 20 mg, 25 mg or 30 mg), about 30-40 mg (e.g., about 30 mg, 35 mg or 40 mg), about 40-50 mg (e.g., about 40 mg, 45 mg or 50 mg), about 50-100 mg (e.g., about 50 mg, 60 mg, 70 mg, 80 mg, 90 mg or 100 mg), about 100-150 mg (e.g., about 100 mg, 110 mg, 120 mg, 130 mg, 140 mg or 150 mg), or about 150-200 mg (e.g., about 150 mg, 160 mg, 170 mg, 180 mg, 190 mg or 200 mg). In some embodiments, the NK-antagonist is administered at 0.25 mg, 1 mg, or 5 mg once a day. In some embodiments, the NK-1 antagonist is administered once a week. In some embodiments, the therapeutically effective dose of the NK-1 antagonist is administered 1, 2, 3 or more times a day, once every two days, once every three days, twice a week or once a week, or as deemed appropriate by the treating physician. In certain embodiments, the therapeutically effective dose of the NK-1 antagonist is administered once or twice daily. It is understood that each therapeutically effective dose of the NK-1 antagonist may be combined with each administration frequency the same as if each and every combination of the therapeutically effective dose and administration frequency were specifically and individually listed.

In some embodiments, about 0.25 to 5 mg or about 5 to 10 mg of the NK-1 antagonist (e.g., serlopitant) is administered once or twice daily. In some embodiments, about 0.25 mg, about 1 mg, about 5 mg or about 10 mg of the NK-1 antagonist (e.g., serlopitant) is administered once or twice daily. In certain embodiments, about 5 mg of the NK-1 antagonist (e.g., serlopitant) is administered once daily.

For treatment of pruritus as described herein, in some embodiments a therapeutically effective amount of the NK-1 antagonist (e.g., serlopitant) is administered over a period of at least about 1 week, 2 weeks, 3 weeks, 4 weeks, 5 weeks, 6 weeks, 2 months, 3 months, 4 months, 5 months, 6 months, 1 year, 2 years, 3 years, 4 years, 5 years or longer (e.g., at least about 1 week, 6 weeks, 3 months, 6 months or 1 year). It is understood that each treatment length may be combined with each therapeutically effective dose and/or administration frequency the same as if each and every combination were specifically and individually listed.

In some embodiments, the NK-1 antagonist (e.g., serlopitant) can also be taken in an irregular manner. For example, the NK-1 antagonist can be administered 1, 2, 3, 4, 5 or more times in a period of 1 week, 2 weeks, 3 weeks or 1 month in an irregular manner. Furthermore, the NK-1 antagonist (e.g., serlopitant) can be taken pro re nata (as needed). For instance, the NK-1 antagonist can be administered 1, 2, 3, 4, 5 or more times, whether in a regular or irregular manner, until pruritus improves. Once relief from itch is achieved, dosing of the NK-1 antagonist can optionally be discontinued. If pruritus returns, administration of the NK-1 antagonist, whether in a regular or irregular manner, can be resumed. The appropriate dosage of, frequency of dosing of and length of treatment with the NK-1 antagonist can be determined by the treating physician.

The NK-1 antagonist (e.g., serlopitant) can be administered via any suitable route. Potential routes of administration of the NK-1 antagonist include without limitation oral, parenteral (including intramuscular, subcutaneous, intradermal, intravascular, intravenous, intraarterial, intraperitoneal, intramedullary, intrathecal and topical), intracavitary, and topical (including dermal/epicutaneous, transdermal, mucosal, transmucosal, intranasal [e.g., by nasal spray or drop], pulmonary [e.g., by oral or nasal inhalation], ocular [e.g., by eye drop], buccal, sublingual, rectal [e.g., by suppository] and vaginal [e.g., by suppository]). In certain embodiments, the NK-1 antagonist is administered orally (e.g., as a capsule or tablet, optionally with an enteric coating). In other embodiments, the NK-1 antagonist is administered parenterally (e.g., intravenously, subcutaneously or intramuscularly). In further embodiments, the NK-1 antagonist is administered topically (e.g., transdermally, transmucosally, pulmonarily, buccally or sublingually).

In some embodiments the NK-1 antagonist (e.g., serlopitant) is administered orally (e.g., as a tablet or capsule) in a dose of about 0.25, 0.5, 1, 5 or 10 mg once daily for at least about 1 week, 2 weeks, 3 weeks, 4 weeks, 5 weeks, 6 weeks, 2 months, 3 months, 4 months, 5 months, 6 months, 1 year, 2 years, 3 years, 4 years, 5 years or longer. The disclosure specifically discloses each of the 44 possible combinations of dose and treatment length. In certain embodiments, about 5 mg of the NK-1 antagonist (e.g., serlopitant) is administered orally (e.g., as a tablet or capsule) once daily for at least about 6 weeks, 3 months, 6 months or 1 year.

In some embodiments where a more rapid establishment of a therapeutic level of the NK-1 antagonist (e.g., serlopitant) is desired, the NK-1 antagonist is administered under a dosing schedule in which a loading dose is administered, followed by (i) one or more additional loading doses and then a plurality of therapeutically effective maintenance doses, or (ii) a plurality of therapeutically effective maintenance doses without an additional loading dose, as deemed appropriate by the treating physician. To establish a therapeutic level of a drug more quickly, a loading dose of the drug is typically larger (e.g., about 1.5, 2, 3, 4 or 5 times larger) than a subsequent maintenance dose. In certain embodiments, the loading dose is about three times larger than the maintenance dose. The therapeutically effective maintenance dose of the NK-1 antagonist can be any therapeutically effective dose described herein, and can be administered in any suitable frequency and for any suitable length of time as described herein. In some embodiments, a loading dose of the NK-1 antagonist (e.g., serlopitant) is administered, followed by administration of a maintenance dose of the NK-1 antagonist after an appropriate time (e.g., after about 12 hr or 24 hr) and thereafter for the duration of therapy—e.g., a loading dose of the NK-1 antagonist is administered on day 1 and a maintenance dose is administered on day 2 and thereafter for the duration of therapy.

In some embodiments, the loading dose of the NK-1 antagonist is about 0.25 to 100 mg, 0.25 to 75 mg, 0.25 to 50 mg, 0.25 to 25 mg, 0.25 to 5 mg, 0.5 to 200 mg, 0.5 to 150 mg, 0.5 to 100 mg, 0.5 to 50 mg, 0.5 to 10 mg, 10 to 20 mg, 20 to 30 mg, 30 to 40 mg, 40 to 50 mg, 50 to 100 mg, 100 to 150 mg or 150 to 200 mg, or as deemed appropriate by the treating physician, which can be administered in a single dose or in divided doses. In certain embodiments, the loading dose of the NK-1 antagonist is about 0.25 to 2.5 mg (e.g., about 0.25 mg, 0.5 mg, 0.75 mg, 1 mg, 2 mg or 2.5 mg), about 0.5 to 5 mg (e.g., about 0.5 mg, 1 mg, 2 mg, 3 mg, 4 mg or 5 mg), about 5 to 10 mg (e.g., about 5 mg, 6 mg, 7 mg, 8 mg, 9 mg or 10 mg), about 10 to 20 mg (e.g., about 10 mg, 15 mg or 20 mg), about 20 to 30 mg (e.g., about 20 mg, 25 mg or 30 mg), about 30 to 40 mg (e.g., about 30 mg, 35 mg or 40 mg), about 40 to 50 mg (e.g., about 40 mg, 45 mg or 50 mg), about 50 to 100 mg (e.g., about 50 mg, 60 mg, 70 mg, 80 mg, 90 mg or 100 mg), about 100 to 150 mg (e.g., about 100 mg, 110 mg, 120 mg, 130 mg, 140 mg or 150 mg), or about 150 to 200 mg (e.g., about 150 mg, 160 mg, 170 mg, 180 mg, 190 mg or 200 mg). In some embodiments, the loading dose of the NK-1 antagonist is at least about 0.5 mg, 1 mg, 2 mg, 3 mg, 4 mg, 5 mg, 6 mg, 7 mg, 8 mg, 9 mg, 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, or 200 mg. In some embodiments, the loading dose of the NK-1 antagonist is less than about 1 mg, 2 mg, 3 mg, 4 mg, 5 mg, 6 mg, 7 mg, 8 mg, 9 mg, 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, or 200 mg.

In some embodiments, the therapeutically effective maintenance dose of the NK-1 antagonist is about 0.25 to 100 mg, 0.25 to 75 mg, 0.25 to 50 mg, 0.25 to 25 mg, 0.25 to 5 mg, 0.5 to 200 mg, 0.5 to 150 mg, 0.5 to 100 mg, 0.5 to 50 mg, 0.5 to 10 mg, 10 to 20 mg, 20 to 30 mg, 30 to 40 mg, 40 to 50 mg, 50 to 100 mg, 100 to 150 mg or 150 to 200 mg, or as deemed appropriate by the treating physician, which can be administered in a single dose or in divided doses. In certain embodiments, the therapeutically effective maintenance dose of the NK-1 antagonist is about 0.25 to 2.5 mg (e.g., about 0.25 mg, 0.5 mg, 0.75 mg, 1 mg, 2 mg or 2.5 mg), about 0.5 to 5 mg (e.g., about 0.5 mg, 1 mg, 2 mg, 3 mg, 4 mg or 5 mg), about 5 to 10 mg (e.g., about 5 mg, 6 mg, 7 mg, 8 mg, 9 mg or 10 mg), about 10 to 20 mg (e.g., about 10 mg, 15 mg or 20 mg), about 20 to 30 mg (e.g., about 20 mg, 25 mg or 30 mg), about 30 to 40 mg (e.g., about 30 mg, 35 mg or 40 mg), about 40 to 50 mg (e.g., about 40 mg, 45 mg or 50 mg), about 50 to 100 mg (e.g., about 50 mg, 60 mg, 70 mg, 80 mg, 90 mg or 100 mg), about 100 to 150 mg (e.g., about 100 mg, 110 mg, 120 mg, 130 mg, 140 mg or 150 mg), or about 150 to 200 mg (e.g., about 150 mg, 160 mg, 170 mg, 180 mg, 190 mg or 200 mg). In some embodiments, the therapeutically effective maintenance dose of the NK-1 antagonist is at least about 0.25 mg, 0.5 mg, 1 mg, 2 mg, 3 mg, 4 mg, 5 mg, 6 mg, 7 mg, 8 mg, 9 mg, 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, or 200 mg. In some embodiments, the therapeutically effective maintenance dose of the NK-1 antagonist is less than about 0.5 mg, 1 mg, 2 mg, 3 mg, 4 mg, 5 mg, 6 mg, 7 mg, 8 mg, 9 mg, 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, or 200 mg. In some embodiments, the therapeutically effective maintenance dose of the NK-1 antagonist is administered once or twice a day, once every two days, once every three days, twice a week or once a week. In some embodiments, the therapeutically effective maintenance dose of the NK-1 antagonist is administered over a period of at least about 1 week, 2 weeks, 3 weeks, 4 weeks, 5 weeks, 6 weeks, 2 months, 3 months, 4 months, 5 months, 6 months, 1 year, 2 years, 3 years, 4 years, or 5 years. It is understood that each treatment length may be combined with each loading dose, maintenance dose, and/or administration frequency the same as if each and every combination were specifically and individually listed.

In some embodiments, the NK-1 antagonist (e.g., serlopitant) is administered in a loading dose of about 0.5-3 mg 3-15 mg or 15-30 mg once or twice on day 1, followed by a maintenance dose of about 0.25-1 mg (e.g., about 0.25, 0.5 or 1 mg), 1-5 mg (e.g., about 1, 3 or 5 mg) or about 5-10 mg (e.g., about 5, 7.5 or 10 mg) once or twice daily for at least about 1 week, 2 weeks, 3 weeks, 4 weeks, 5 weeks, 6 weeks, 2 months, 3 months, 4 months, 5 months, 6 months, 1 year, 2 years, 3 years, 4 years, 5 years or longer, where the loading dose is about three times larger than the maintenance dose and is administered orally (e.g., as a tablet or capsule). In certain embodiments, the NK-1 antagonist (e.g., serlopitant) is administered in a loading dose of about 15 mg orally (e.g., as a tablet) on day 1, followed by a maintenance dose of about 5 mg orally (e.g., as a tablet) once daily, optionally at bedtime, for at least about 6 weeks, 3 months, 6 months, about 1 year or longer.

In other embodiments, a first loading dose of the NK-1 antagonist (e.g., serlopitant) is administered on day 1, a second loading dose is administered on day 2, and a maintenance dose is administered on day 3 and thereafter for the duration of therapy. In certain embodiment, the first loading dose is about three times larger than the maintenance dose, and the second loading dose is about two times larger than the maintenance dose.

The NK-1 antagonist (e.g., serlopitant) can be administered at any time convenient to the patient. NK-1 antagonists may cause drowsiness. To avoid or minimize drowsiness during the day, the NK-1 antagonist can be administered shortly before the patient goes to bed. Moreover, use of the NK-1 antagonist at night can aid with sleep and decrease nocturnal itch and scratching. Accordingly, in certain embodiments the NK-1 antagonist (e.g., serlopitant) is administered at bedtime (e.g., once daily at bedtime). The NK-1 antagonist (e.g., serlopitant) can also be administered at any appropriate time during the day or awake hours (e.g., in the morning).

In some embodiments, the NK-1 antagonist (e.g., serlopitant) is administered without food. In some embodiments, the NK-1 antagonist (e.g., serlopitant) is administered at least about 1 or 2 hours before or after a meal at any time of the day. In certain embodiments, the NK-1 antagonist is administered at least about 2 hours after an evening meal, or at least about 2 hours before or after a meal in the morning. The NK-1 antagonist (e.g., serlopitant) can also be taken substantially concurrently with food, such as within about 1 hour, 30 minutes or 15 minutes before or after a meal, or with a meal, at any time of the day.

In some embodiments, the pruritus is pruritus in the elderly (over 40 years of age). In other embodiments, the pruritus is chronic idiopathic pruritus. In other embodiments, the pruritus is chronic pruritus of unknown origin. In additional embodiments, the pruritus is refractory or resistant to other antipruritic therapies without an NK-1 antagonist.

In some embodiments, one or any combination, or all, of the following apply:

i) the duration of the pruritus is about one year or longer;

ii) the subject is about 40 years of age or older;

iii) the subject has no inflammatory skin disease; and

iv) the pruritus is idiopathic.

In some embodiments, one or any combination, or all, of the following apply:

i) the duration of the pruritus is about one year or longer;

ii) the subject is about 18 years of age or older;

iii) the subject has no inflammatory skin disease; and

iv) the pruritus is idiopathic.

In some embodiments, one or any combination, or all, of the following apply:

i) the duration of the pruritus is about one year or longer;

ii) the subject is about 40 years of age or older;

iii) the subject has no inflammatory skin disease; and

iv) the pruritus is of unknown origin.

In some embodiments, one or any combination, or all, of the following apply:

i) the duration of the pruritus is about one year or longer;

ii) the subject is about 18 years of age or older;

iii) the subject has no inflammatory skin disease; and

iv) the pruritus is of unknown origin.

An NK-1 antagonist (e.g., serlopitant) can be used to treat pruritus of any degree of severity (e.g., mild, moderate or severe). In addition to its antipruritic effect (e.g., blocking NK1-expressing spinal projection neurons from transmitting itch signals to the brain), an NK-1 antagonist (e.g., serlopitant) can have other beneficial properties that aid in alleviating pruritus, such as anti-inflammatory, anti-proliferative and anti-metastatic properties.

The disclosure provides an NK-1 antagonist (e.g., serlopitant), or a composition comprising an NK-1 antagonist (e.g., serlopitant), for use in the treatment of pruritus, wherein one or more of the following conditions apply: i) the duration of the pruritus is about one year or longer: ii) the subject is about 40 years of age or older; iii) the subject has no inflammatory skin disease; and (iv) the pruritus is idiopathic. The disclosure further provides an NK-1 antagonist (e.g., serlopitant), or a composition comprising an NK-1 antagonist (e.g., serlopitant), for use in the treatment of pruritus, wherein one or any combination, or all, of the following conditions apply: i) the duration of the pruritus is about one year or longer; ii) the subject is about 40 years of age or older; iii) the subject has no inflammatory skin disease; and (iv) the pruritus is of unknown origin. The disclosure further provides for the use of an NK-1 antagonist (e.g., serlopitant) in the preparation of a medicament for the treatment of pruritus, wherein one or more of the following conditions apply: i) the duration of the pruritus is about one year or longer; ii) the subject is about 40 years of age or older; iii) the subject has no inflammatory skin disease; and (iv) the pruritus is idiopathic. The disclosure further provides for the use of an NK-1 antagonist (e.g., serlopitant) in the preparation of a medicament for the treatment of pruritus, wherein one or any combination, or all, of the following conditions apply: i) the duration of the pruritus is about one year or longer: ii) the subject is about 40 years of age or older; iii) the subject has no inflammatory skin disease; and (iv) the pruritus is of unknown origin.

### Neurokinin-1 Antagonists

As described above, the disclosure provides for the use of an NK-1 antagonist in the treatment of pruritus, wherein one or more of the following conditions apply: i) the duration of the pruritus is about one year or longer; ii) the subject is about 40 years of age or older; iii) the subject has no inflammatory skin disease; and (iv) the pruritus is idiopathic. The disclosure further provides for the use of an NK-1 antagonist in the treatment of pruritus, wherein one or any combination, or all, of the following conditions apply: i) the duration of the pruritus is about one year or longer; ii) the subject is about 40 years of age or older; iii) the subject has no inflammatory skin disease; and (iv) the pruritus is of unknown origin. Examples of NK-1 antagonists include without limitation aprepitant (L-754030 or MK-(0)869), fosaprepitant (L-758298), befetupitant, burapitant (SSR-240600), casopitant (GW-679769), dapitant (RPR-100893), ezlopitant (CJ-11974), figopitant (BIIF-1149), lanepitant (LY-303870), maropitant (CJ-11972), netupitant, fosnetupitant, nolpitantium (SR-140333), orvepitant (GW-823296), rolapitant (SCH-619734), SCH-720881 (active metabolite of rolapitant), serlopitant (MK-(0)594 or VPD-737), tradipitant (VLY-686 or LY-686017), vestipitant (GW-597599), vofopitant (GR-205171), hydroxyphenyl propamidobenzoic acid, maltooligosaccharides (e.g., maltotetraose and maltopentaose), spantides (e.g., spantide 1 and 11), AV-608, AV-818, AZD-2624, CGP-49823, CJ-17493, CP-96345, CP-99994, CP-122721, DNK-333 (NK1/NK2), FK-224 (NK1/NK2), FK-888, GR-82334, GR-203040, GR-205171. GSK-424887, HSP-117, KRP-103, L-703606, L-733060, L-736281, L-759274, L-760735, LY-686017, M516102, MDL-105212 (NK1/NK2), MK-0303 (L-001182885), MK-8478 (L-001983867), NKP-608, PD-154075, R-116031, R-116301, RP-67580, S-41744, SCH-206272 (NK-1/NK-2/NK-3), SCH-388714, SCH-900978, SLV-317, T-2328, TA-5538, TAK-637, TKA-731, WIN-51708, ZD-4974, ZD-6021 (NK1/NK2), and analogs, derivatives, prodrugs, metabolites, salts and stereoisomers thereof.

In some embodiments, the NK-1 antagonist is a selective NK-1 antagonist. In certain embodiments, the NK-1 antagonist is serlopitant or a pharmaceutically acceptable salt, solvate, hydrate, clathrate, polymorph, prodrug, metabolite or stereoisomer thereof.

In other embodiments, the NK-1 antagonist is aprepitant or fosaprepitant, or a pharmaceutically acceptable salt, solvate, hydrate, clathrate, polymorph, prodrug, metabolite or stereoisomer thereof. In additional embodiments, the NK-1 antagonist is befetupitant or a pharmaceutically acceptable salt, solvate, hydrate, clathrate, polymorph, prodrug, metabolite or stereoisomerthereof. In certain embodiments, the NK-1 antagonist is burapitant or a pharmaceutically acceptable salt, solvate, hydrate, clathrate, polymorph, prodrug, metabolite or stereoisomer thereof. In further embodiments, the NK-1 antagonist is casopitant or a pharmaceutically acceptable salt, solvate, hydrate, clathrate, polymorph, prodrug, metabolite or stereoisomerthereof. In still further embodiments, the NK-1 antagonist is dapitant or a pharmaceutically acceptable salt, solvate, hydrate, clathrate, polymorph, prodrug, metabolite or stereoisomer thereof. In yet further embodiments, the NK-1 antagonist is ezlopitant or a pharmaceutically acceptable salt, solvate, hydrate, clathrate, polymorph, prodrug, metabolite or stereoisomer thereof. In certain embodiments, the NK-1 antagonist is figopitant or a pharmaceutically acceptable salt, solvate, hydrate, clathrate, polymorph, prodrug, metabolite or stereoisomer thereof. In other embodiments, the NK-1 antagonist is lanepitant or a pharmaceutically acceptable salt, solvate, hydrate, clathrate, polymorph, prodrug, metabolite or stercoisomer thereof. Instill other embodiments, the NK-1 antagonist is maropitant or a pharmaceutically acceptable salt, solvate, hydrate, clathrate, polymorph, prodrug, metabolite or stereoisomer thereof.

In additional embodiments, the NK-1 antagonist is netupitant or fosnetupitant, or a pharmaceutically acceptable salt, solvate, hydrate, clathrate, polvmorph, prodrug, metabolite or stereoisomer thereof. In further embodiments, the NK-1 antagonist is nolpitantium or a pharmaceutically acceptable salt, solvate, hydrate, clathratc, polymorph, prodrug, metabolite or stereoisomer thereof. Instill further embodiments, the NK-1 antagonist is orvepitant or a pharmaceutically acceptable salt, solvate, hydrate, clathrate, polymorph, prodrug, metabolite or stereoisomer thereof. In yet further embodiments, the NK-1 antagonist is rolapitant or SCH-720881 (active metabolite of rolapitant), or a pharmaceutically acceptable salt, solvate, hydrate, clathrate, polvmorph, prodrug, metabolite or stereoisomer thereof. In other embodiments, the NK-1 antagonist is tradipitant or a pharmaceutically acceptable salt, solvate, hydrate, clathrate, polymorph, prodrug, metabolite or stereoisomer thereof. Instill other embodiments, the NK-1 antagonist is vestipitant or a pharmaceutically acceptable salt, solvate, hydrate, clathrate, polymorph, prodrug, metabolite or stereoisomer thereof. In yet other embodiments, the NK-1 antagonist is vofopitant or a pharmaceutically acceptable salt, solvate, hydrate, clathrate, polymorph, prodrug, metabolite or stereoisomer thereof.

In some embodiments, the NK-1 antagonist is not aprepitant or fosaprepitant.

### Description of Serlopitant

Serlopitant is a potent and highly selective antagonist of neurokinin-1 (also called substance P receptor). By binding to and not activating NK-1, serlopitant can block actions of substance P, including transmission of itch signals to the brain, elicitation of inflammation, stimulation of growth of cancer cells, and promotion of metastasis of cancer cells.

Serlopitant has the structure shown below. The IUPAC name for serlopitant is 3-[(3aR,4R,5S,7aS)-5-[(1R)-1-[3,5-bis(trifluoromethyl)phenyl]cthoxy]-4-(4-fluorophenyI)-1,3,3a,4,5,6,7,7a-octahydroisoindol-2-yl]cyclopent-2-en-1-one. The USAN name for serlopitant is 3-[(3aR,4R,5S,7aS)-5-[(1R)-1-[3,5-bis(trifluoromethyl)phenyl]ethoxyl-4-(4-fluorophenyl)octahydro-2H-isoindol-2-yl]cyclopent-2-en-1-one. The disclosure also encompasses all stereoisomers of serlopitant, including both enantiomers and all diasteromers of serlopitant in substantially pure form and mixtures of both enantiomers (including a racemic mixture) and mixtures of two or more diastereomers of serlopitant in any ratio. The disclosure further encompasses all isotopically enriched forms of serlopitant, including without limitation those enriched in the content of ²H (deuterium), ¹³C, ¹⁵N, ¹⁷O, ¹⁹O or ¹⁹F, or any combination thereof, at one or more, or all, locations of the corresponding atom(s). Moreover, the disclosure encompasses any and all salt forms of serlopitant. Various methods of synthesizing serlopitant are known in the art. See, e.g., Jiang et al., J. Med. Chem., 52:3039-3046 (2009); U.S. Pat. No. 7,544,815 by Kuethe et al.: and U.S. Pat. No. 7,217,731 by Bunda et a.

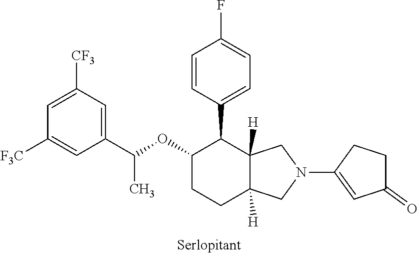

Whether as a free base or a salt, serlopitant can exist unsolvated or unhydrated, or solvated or hydrated. Solvated forms of serlopitant can be formed with a pharmaceutically acceptable solvent, such as water or ethanol. In certain embodiments, serlopitant, whether as a free base or a salt, is used substantially unhydrated.

The disclosure also encompasses polymorphs (crystalline forms) of serlopitant. Examples of polymorphs of serlopitant include without limitation anhydrous crystalline Forms I and II of free base serlopitant as disclosed in US Pub. 2009/0270477 by Kuethe et al. Form I is characterized by diffraction peaks obtained from X-ray powder diffraction pattern corresponding to d-spacings of 10.4, 9.9, 9.2, 5.5, 5.0, 4.1, 3.9, 3.6 and 3.5 angstroms. Form II is characterized by diffraction peaks obtained from X-ray powder diffraction pattern corresponding to d-spacings of 7.7, 5.3, 4.9, 4.8, 4.6, 4.2, 3.9, 3.8 and 2.8 angstroms. Form I is thermodynamically more stable below 70° C. and is non-hygroscopic under all tested relative humidity conditions. In certain embodiments, serlopitant is used in the form of polymorph Form I.

### Stereoisomers

The present disclosure encompasses all possible stereoisomers, including both enantiomers and all possible diastereomers in substantially pure form and mixtures of both enantiomers in any ratio (including a racemic mixture of enantiomers) and mixtures of two or more diastereomers in any ratio, of the compounds described herein, including without limitation neurokinin-1 antagonists, and not only the specific stereoisomers as indicated by drawn structure or nomenclature Some embodiments of the disclosure relate to the specific stereoisomers indicated by drawn structure or nomenclature. If the phrase “or stereoisomers thereof” or the like with respect to a compound is recited in certain instances of the disclosure, such recitation shall not be interpreted as an intended omission of any of the other possible stereoisomers of the compound in other instances of the disclosure where the compound is mentioned without recitation of the phrase “or stereoisomers thereof” or the like, unless stated otherwise or the context clearly indicates otherwise.

### Salt Forms of Drug Substances

Drug substances (e.g., NK-1 antagonists such as serlopitant) may exist in a non-salt form (e.g., a free base or a free acid, or having no basic or acidic atom or functional group) or as salts if they can form salts. Drug substances that can form salts can be used in the non-salt form or in the form of pharmaceutically acceptable salts. If a drug has. e.g., a basic nitrogen atom, the drug can form an addition salt with an acid (e.g., a mineral acid [such as HCl, HBr, HI, nitric acid, phosphoric acid or sulfuric acid] or an organic acid [such as a carboxylic acid or a sulfonic acid]). Suitable acids for use in the preparation of pharmaceutically acceptable salts include without limitation acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, D-gluconic acid, D-glucuronic acid, L-glutamic acid, alpha-oxo-glutaric acid, glycolic acid, hippuric acid, hydrobromic acid, hydrochloric acid, hydroiodic acid, (±)-DL-lactic acid, (+)-L-lactic acid, lactobionic acid, lauric acid, maleic acid, (−)-L-malic acid, malonic acid, (+)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, propionic acid, L-pyroglutamic acid, pyruvic acid, saccharic acid, salicylic acid, 4-amino-salicylic acid, sebacic acid, stearic acid, succinic acid, sulfuric acid, tannic acid, (t)-DL-tartaric acid, (+)-L-tartaric acid, thiocyanic acid, p-toluenesulfonic acid, undecylenic acid, and valeric acid.

If a drug has an acidic group (e.g., a carboxyl group), the drug can form an addition salt with a base. Pharmaceutically acceptable base addition salts can be formed with, e.g., metals (e.g., alkali metals or alkaline earth metals) or amines (e.g., organic amines). Non-limiting examples of metals useful as cations include alkali metals (e.g., lithium, sodium, potassium and cesium), alkaline earth metals (e.g., magnesium and calcium), aluminum and zinc. Metal cations can be provided by way of, e.g., inorganic bases, such as hydroxides, carbonates and hydrogen carbonates. Non-limiting examples of organic amines useful for forming base addition salts include chloroprocaine, choline, cyclohexylamine, dibenzylamine. N,N′-dibenzylethylenediamine, dicyclohexylamine, diethanolamine, ethylenediamine, N-ethylpiperidine, histidine, isopropylamine, N-methylglucamine, procaine, pyrazine, triethylamine and trimethylamine. Pharmaceutically acceptable salts are discussed in detail in Handbook of Pharmaceutical Salts, Properties, Selection and Use, P. Stahl and C. Wermuth, Eds., Wiley-VCH (2011).

### Pharmaceutical Compositions

To treat chronic pruritus, an NK-1 antagonist (e.g., serlopitant) can be administered alone or in the form of a pharmaceutical composition. In some embodiments, a pharmaceutical composition comprises an NK-1 antagonist (e.g., serlopitant) or a pharmaceutically acceptable salt, solvate, hydrate, clathrate, polymorph, prodrug, metabolite or stereoisomer thereof, and one or more pharmaceutically acceptable carriers or excipients. The composition can optionally contain an additional therapeutic agent described herein. In general, a pharmaceutical composition contains a therapeutically effective amount of a therapeutic agent (e.g., an NK-1 antagonist) or an appropriate fraction thereof and one or more pharmaceutically acceptable carriers or excipients, and is formulated for administration to a subject for therapeutic use. For purposes of the content of a pharmaceutical composition, the terms “therapeutic agent”, “active ingredient”, “active agent” and “drug” encompass prodrugs.

A pharmaceutical composition contains a therapeutic agent (e.g., an NK-1 antagonist) in substantially pure form. In some embodiments, the purity of the therapeutic agent is at least about 95%, 96%, 97%, 98% or 99%. In certain embodiments, the purity of the therapeutic agent is at least about 98% or 99%. In addition, a pharmaceutical composition is substantially free of contaminants or impurities. In some embodiments, the level of contaminants or impurities other than residual solvent in a pharmaceutical composition is no more than about 5%, 4%, 3%, 2% or 1% relative to the combined weight of the intended active and inactive ingredients. In certain embodiments, the level of contaminants or impurities other than residual solvent in a pharmaceutical composition is no more than about 2% or 1% relative to the combined weight of the intended active and inactive ingredients. Pharmaceutical compositions generally are prepared according to current good manufacturing practice (GMP), as recommended or required by, e.g., the Federal Food, Drug, and Cosmetic Act § 501(a)(2)(B) and the International Conference on Harmonisation Q7 Guideline.

Pharmaceutical compositions/formulations can be prepared in sterile form. For example, pharmaceutical compositions/formulations for parenteral administration by injection or infusion generally are sterile. Sterile pharmaceutical compositions/formulations are compounded or manufactured according to pharmaceutical-grade sterilization standards known to those of skill in the art, such as those disclosed in or required by the United States Pharmacopeia Chapters 797, 1072 and 1211, and 21 Code of Federal Regulations 211.

Pharmaceutically acceptable carriers and excipients include pharmaceutically acceptable vehicles, substances and materials. Non-limiting examples of types of excipients include liquid and solid fillers, diluents, binders, lubricants, glidants, solubilizers, surfactants, dispersing agents, disintegration agents, emulsifying agents, wetting agents, suspending agents, thickeners, solvents, isotonic agents, buffers, pH adjusters, absorption-delaying agents, stabilizers, antioxidants, preservatives, antimicrobial agents, antibacterial agents, antifungal agents, chelating agents, adjuvants, sweetening agents, flavoring agents, coloring agents, encapsulating materials and coating materials. The use of such excipients in pharmaceutical formulations is known in the art. For example, conventional vehicles and carriers include without limitation oils (e.g., vegetable oils such as olive oil and sesame oil), aqueous solvents {e.g., saline, buffered saline (e.g., phosphate-buffered saline [PBS]) and isotonic solutions (e.g., Ringer's solution)), and organic solvents (e.g., dimethyl sulfoxide [DMSO] and alcohols [e.g., ethanol, glycerol and propylene glycol]). Except insofar as any conventional carrier or excipient is incompatible with the active ingredient, the disclosure encompasses the use of conventional carriers and excipients in formulations containing a therapeutic agent (e.g., an NK-1 antagonist). See, e.g., Remington: The Science and Practice of Pharmacy, 21st Ed., Lippincott Williams & Wilkins (Philadelphia, Pa.) (2005); Handbook of Pharmaceutical Excipients, 5th Ed., Rowe et al., Eds., The Pharmaceutical Press and the American Pharmaceutical Association (2005); Handbook of Pharmaceutical Additives, 3rd Ed., Ash and Ash, Eds., Gower Publishing Co. (2007); and Pharmaceutical Preformulation and Formulation, Gibson, Ed., CRC Press (Boca Raton, Fla.) (2004).

Proper formulation can depend on various factors, such as the route of administration chosen. Potential routes of administration of pharmaceutical compositions comprising a therapeutic agent (e.g., an NK-1 antagonist) include without limitation oral, parenteral (including intramuscular, subcutaneous, intradermal, intravascular, intravenous, intraarterial, intraperitoneal, intramedullary, intrathecal and topical), intracavitary, and topical (including dermal/epicutaneous, transdermal, mucosal, transmucosal, intranasal [e.g., by nasal spray or drop], pulmonary [e.g., by oral or nasal inhalation], ocular [e.g., by eye drop], buccal, sublingual, rectal [e.g., by suppository] and vaginal [e.g., by suppository]).

As an example, formulations of an NK-1 antagonist (e.g., serlopitant) suitable for oral administration can be presented as, e.g., boluses; tablets, capsules, pills, cachets or lozenges; as powders or granules; as semisolids, electuaries, pastes or gels; as solutions or suspensions in an aqueous liquid or/and a non-aqueous liquid; or as oil-in-water liquid emulsions or water-in-oil liquid emulsions.

Tablets can contain an NK-1 antagonist (e.g., serlopitant) in admixture with, e.g., a filler or inert diluent (e.g., calcium carbonate, calcium phosphate, lactose, mannitol or microcrystalline cellulose), a binding agent (e.g., a starch, gelatin, acacia, alginic acid or a salt thereof, or microcrystalline cellulose), a lubricating agent (e.g., stearic acid, magnesium stearate, talc or silicon dioxide), and a disintegrating agent (e.g., crospovidone, croscarmellose sodium or colloidal silica), and optionally a surfactant (e.g., sodium lauryl sulfate). The tablets can be uncoated or can be coated with, e.g., an enteric coating that protects the active ingredient from the acidic environment of the stomach, or with a material that delays disintegration and absorption of the active ingredient in the gastrointestinal tract and thereby provides a sustained action over a longer time period. In certain embodiments, a tablet comprises an NK-1 antagonist (e.g., serlopitant), mannitol, microcrystalline cellulose, magnesium stearate, silicon dioxide, croscarmellose sodium and sodium lauryl sulfate, and optionally lactose monohydrate, and the tablet is optionally film-coated (e.g., with Opadry®).

Push-fit capsules or two-piece hard gelatin capsules can contain an NK-1 antagonist (e.g., serlopitant) in admixture with, e.g., a filler or inert solid diluent (e.g., calcium carbonate, calcium phosphate, kaolin or lactose), a binder (e.g., a starch), a glidant or lubricant (e.g., talc or magnesium stearate), and a disintegrant (e.g., crospovidone), and optionally a stabilizer or/and a preservative. For soft capsules or single-piece gelatin capsules, an NK-1 antagonist (e.g., serlopitant) can be dissolved or suspended in a suitable liquid (e.g., liquid polyethylene glycol or an oil medium, such as a fatty oil, peanut oil, olive oil or liquid paraffin), and the liquid-filled capsules can contain one or more other liquid excipients or/and semi-solid excipients, such as a stabilizer or/and an amphiphilic agent (e.g., a fatty acid ester of glycerol, propylene glycol or sorbitol).

Compositions for oral administration can also be formulated as solutions or suspensions in an aqueous liquid or/and a non-aqueous liquid, or as oil-in-water liquid emulsions or water-in-oil liquid emulsions. Dispersible powder or granules of an NK-1 antagonist (e.g., serlopitant) can be mixed with any suitable combination of an aqueous liquid, an organic solvent or/and an oil and any suitable excipients (e.g., any combination of a dispersing agent, a wetting agent, a suspending agent, an emulsifying agent or/and a preservative) to form a solution, suspension or emulsion.

In some embodiments, an NK-1 antagonist (e.g., serlopitant) is contained in an amphiphilic vehicle of a liquid or semi-solid formulation for oral administration which provides improved solubility, stability and bioavailability of the NK-1 antagonist, as described in US Pub. 2010/0209496 by Dokou et al. The amphiphilic vehicle contains a solution, suspension, emulsion (e.g., oil-in-water emulsion) or semi-solid mixture of the NK-1 antagonist (e.g., serlopitant) admixed with liquid or/and semi-solid excipients which fills an encapsulated dosage form (e.g., a hard gelatin capsule or a soft gelatin capsule containing a plasticizer [e.g., glycerol or/and sorbitol]). In some embodiments, the amphiphilic vehicle comprises an amphiphilic agent selected from fatty acid esters of glycerol (glycerin), propylene glycol and sorbitol. In certain embodiments, the amphiphilic agent is selected from mono- and di-glycerides of C₈-C₁₂ saturated fatty acids. In further embodiments, the amphiphilic agent is selected from CAPMUL® MCM, CAPMUL® MCM 8, CAPMUL® MCM 10, IMWITOR® 308, IMWITOR® 624, IMWITOR® 742, IMWITOR® 988, CAPRYOL® PGMC, CAPRYOL® 90, LAUROGLYCOL™ 90, CAPTEX® 200, CRILL™ 1, CRILL™ 4, PECEOL® and MAISINE® 35-1. In some embodiments, the amphiphilic vehicle further comprises propylene glycol, a propylene glycol-sparing agent (e.g., ethanol or/and glycerol), or an antioxidant (e.g., butylated hydroxyanisole, butylated hydroxytoluene, propyl gallate or/and sodium sulfite), or any combination or all thereof. In additional embodiments, the amphiphilic vehicle contains on a weight basis about 0.1-5% of the NK-1 antagonist (e.g., serlopitant), about 50-90% of the amphiphilic agent, about 5-40% of propylene glycol, about 5-20% of the propylene glycol-sparing agent, and about 0.01-0.5% of the antioxidant.

An NK-1 antagonist (e.g., serlopitant) can also be formulated for parenteral administration by injection or infusion to circumvent gastrointestinal (GI) absorption and first-pass metabolism. A representative parenteral route is intravenous. Additional advantages of intravenous administration include direct administration of a therapeutic agent into systemic circulation to achieve a rapid systemic effect, and the ability to administer the agent continuously or/and in a large volume if desired. Formulations for injection or infusion can be in the form of, e.g., solutions, suspensions or emulsions in oily or aqueous vehicles, and can contain excipients such as suspending agents, dispersing agents or/and stabilizing agents. For example, aqueous or non-aqueous (e.g., oily) sterile injection solutions can contain an NK-1 antagonist (e.g., serlopitant) along with excipients such as an antioxidant, a buffer, a bacteriostat and solutes that render the formulation isotonic with the blood of the subject. Aqueous or non-aqueous sterile suspensions can contain an NK-1 antagonist (e.g., serlopitant) along with excipients such as a suspending agent and a thickening agent, and optionally a stabilizer and an agent that increases the solubility of the NK-1 antagonist to allow for the preparation of a more concentrated solution or suspension. As another example, a sterile aqueous solution for injection or infusion (e.g., subcutaneously or intravenously) can contain an NK-1 antagonist (e.g., serlopitant), NaCl, a buffering agent (e.g., sodium citrate), a preservative (e.g., meta-cresol), and optionally a base (e.g., NaOH) or/and an acid (e.g., HCl) to adjust pH.

In some embodiments, an NK-1 antagonist (e.g., serlopitant) is delivered from a sustained-release composition. As used herein, the term “sustained-release composition” encompasses sustained-release, prolonged-release, extended-release, slow-release and controlled-release compositions, systems and devices. Use of a sustained-release composition can have benefits, such as an improved profile of the amount of the drug delivered to the target site(s) over a time period, including delivery of a therapeutically effective amount of the drug over a prolonged time period. In certain embodiments, a sustained-release composition delivers an NK-1 antagonist over a period of at least about 2 days, 3 days, 1 week, 2 weeks, 3 weeks, 1 month, 2 months, 3 months or longer. In some embodiments, a sustained-release composition is a drug-encapsulation system, such as, e.g., nanoparticles, microparticles or a capsule made of, e.g., a biodegradable polymer or/and a hydrogel. In certain embodiments, a sustained-release composition comprises a hydrogel. Non-limiting examples of polymers of which a hydrogel can be composed include polyvinyl alcohol, acrylate polymers (e.g., sodium polyacrylate), and other homopolymers and copolymers having a relatively large number of hydrophilic groups (e.g., hydroxyl or/and carboxylate groups). In other embodiments, a sustained-release drug-encapsulation system comprises a membrane-enclosed reservoir, wherein the reservoir contains a drug and the membrane is permeable to the drug. Such a drug-delivery system can be in the form of, e.g., a transdermal patch.

In certain embodiments, a sustained-release composition is an oral dosage form, such as a tablet or capsule. For example, a drug can be embedded in an insoluble porous matrix such that the dissolving drug must make its way out of the matrix before it can be absorbed through the GI tract. Alternatively, a drug can be embedded in a matrix that swells to form a gel through which the drug exits Sustained release can also be achieved by way of a single-layer or multi-layer osmotic controlled-release oral delivery system (OROS). An OROS is a tablet with a semi-permeable outer membrane and one or more small laser-drilled holes in it. As the tablet passes through the body, water is absorbed through the semi-permeable membrane via osmosis, and the resulting osmotic pressure pushes the drug out through the hole(s) in the tablet and into the GI tract where it can be absorbed.

In further embodiments, a sustained-release composition is formulated as polymeric nanoparticles or microparticles, which can be delivered, e.g., by injection or inhalation or as an implant (e.g., a depot). In some embodiments, the polymeric implant or polymeric nanoparticles or microparticles are composed of a biodegradable polymer. In certain embodiments, the biodegradable polymer comprises lactic acid or/and glycolic acid [e.g., an L-lactic acid-based copolymer, such as poly(L-lactide-co-glycolide) or poly(L-lactic acid-co-D,L-2-hydroxyoctanoic acid)]. For instance, biodegradable polymeric microspheres composed of polylactic acid or/and polyglycolic acid can serve as sustained-release pulmonary drug-delivery systems. The biodegradable polymer of the polymeric implant or polymeric nanoparticles or microparticles can be selected so that the polymer substantially completely degrades around the time the period of treatment is expected to end, and so that the byproducts of the polymer's degradation, like the polymer, are biocompatible.

For a delayed or sustained release of an NK-1 antagonist (e.g., serlopitant), a composition can also be formulated as a depot that can be implanted in or injected into a subject, e.g., intramuscularly or subcutaneously. A depot formulation can be designed to deliver an NK-1 antagonist over a longer period of time, e.g., over a period of at least about 1 week, 2 weeks, 3 weeks, 1 month, 6 weeks, 2 months, 3 months or longer. For example, an NK-1 antagonist (e.g., serlopitant) can be formulated with a polymeric material (e.g., polyethylene glycol [PEG], polylactic acid [PLA] or polyglycolic acid [PGA], or a copolymer thereof [e.g., PLGAJ], a hydrophobic material (e.g., as an emulsion in an oil) or/and an ion-exchange resin, or as a sparingly soluble derivative (e.g., a sparingly soluble salt). As an illustrative example, an NK-1 antagonist (e.g., serlopitant) can be incorporated or embedded in sustained-release microparticles composed of PLGA and formulated as a monthly depot.

An NK-1 antagonist (e.g., serlopitant) can also be contained or dispersed in a matrix material. The matrix material can comprise a polymer (e.g., ethylene-vinyl acetate) and controls release of the drug by controlling dissolution or/and diffusion of the drug from, e.g., a reservoir, and can enhance the stability of the drug while contained in the reservoir. Such a release system can be designed as a sustained-release system, can be configured as, e.g., a transdermal or transmucosal patch, and can contain an excipient that can accelerate the drug's release, such as a water-swellable material (e.g., a hydrogel) that aids in expelling the drug out of the reservoir. U.S. Pat. Nos. 4,144,317 and 5,797,898 describe examples of such a release system.

The release system can provide a temporally modulated release profile (e.g., pulsatile release) when time variation in plasma levels is desired, or a more continuous or consistent release profile when a constant plasma level is desired. Pulsatile release can be achieved from an individual reservoir or from a plurality of reservoirs. For example, where each reservoir provides a single pulse, multiple pulses (“pulsatile” release) are achieved by temporally staggering the single pulse release from each of multiple reservoirs. Alternatively, multiple pulses can be achieved from a single reservoir by incorporating several layers of a release system and other materials into a single reservoir. Continuous release can be achieved by incorporating a release system that degrades, dissolves, or allows diffusion of a compound through it over an extended time period. In addition, continuous release can be approximated by releasing several pulses of a compound in rapid succession (“digital” release). An active release system can be used alone or in conjunction with a passive release system, as described in U.S. Pat. No. 5,797,898.

In addition, pharmaceutical compositions comprising an NK-1 antagonist (e.g., serlopitant) can be formulated as, e.g., liposomes, micelles (e.g., those composed of biodegradable natural or/and synthetic polymers, such as lactosomes), nanoparticles, microparticles or microspheres, whether or not designed for sustained release. For example, liposomes can be used as a sustained-release pulmonary drug-delivery system that delivers a drug to the alveolar surface for treatment of a systemic disorder.

The pharmaceutical compositions can be manufactured in any suitable manner known in the art, e.g., by means of conventional mixing, dissolving, suspending, granulating, dragee-making, levigating, emulsifying, encapsulating, entrapping or compressing processes.

A pharmaceutical composition can be presented in unit dosage form as a single dose wherein all active and inactive ingredients are combined in a suitable system, and components do not need to be mixed to form the composition to be administered. The unit dosage form can contain an effective dose, or an appropriate fraction thereof, of a drug (e.g., an NK-1 antagonist). Representative examples of a unit dosage form include a tablet, capsule or pill for oral administration, and a single-use pen comprising a pre-filled syringe, a needle and a needle cover for parenteral (e.g., intravenous or subcutaneous) injection of the drug.

Alternatively, a pharmaceutical composition can be presented as a kit in which the therapeutic agent, excipients and carriers (e.g., solvents) are provided in two or more separate containers (e.g., ampules, vials, tubes, bottles or syringes) and need to be combined to form the composition to be administered. The kit can contain instructions for storing, preparing and administering the composition (e.g., a solution to be injected intravenously or subcutaneously).

A kit can contain all active and inactive ingredients in unit dosage form or the active ingredient and inactive ingredients in two or more separate containers, and can contain instructions for administering or using the pharmaceutical composition to treat chronic pruritus.

In some embodiments, a kit contains an NK-1 antagonist (e.g., serlopitant) or a pharmaceutical composition comprising the same, and instructions for administering or using the NK-1 antagonist or the pharmaceutical composition comprising the same to treat chronic pruritus.

### Representative Embodiments

The following embodiments of the disclosure are provided by way of example:
1. A method of treating a pruritus in a subject, comprising administering to the subject a therapeutically effective amount of a neurokinin-1 (NK-1) antagonist, wherein any one or more of i)-iv) apply:
   i) the duration of the pruritus is about six months or longer;
   ii) the subject is about 40 years of age or older;
   iii) the subject has no inflammatory skin disease, and
   iv) the pruritus is idiopathic.
2. The method of embodiment 1, wherein the pruritus has a duration of at least about 1 year, 2 year, 3 years or 5 years.
3. The method of embodiment 1 or 0, wherein the chronic pruritus has a duration of at least about 7 years, 10 years or 15 years.
4. The method of any one of the preceding embodiments, wherein the pruritus is characterized by sensitization or hypersensitization of the central nervous system (e.g., dorsal root ganglion neurons, spinal dorsal horn neurons or spinal trigeminal nucleus [e.g., medullary dorsal horn] neurons, or any combination or all thereof).
5. The method of any one of the preceding embodiments, wherein the pruritus is characterized by sensitization or hypersensitization of the peripheral nervous system (e.g., unmyelinated C fibers, thinly myelinated A6 fibers or myelinated AP fibers, or any combination or all thereof).
6. The method of any one of the preceding embodiments, wherein the pruritus is characterized by spontaneous itch, alloknesis or hyperknesis, or any combination or all thereof.
7. The method of embodiment 6, wherein the NK-1 antagonist inhibits spontaneous itch, alloknesis or hyperknesis, or any combination or all thereof.
8. The method of embodiment 7, wherein the NK-1 antagonist reduces the frequency or/and the intensity of spontaneous itch, alloknesis or hyperknesis, or any combination or all thereof, by at least about 20%, 30%, 40%, 50%, 60%, 70%, 80%, 90% or 95% (e.g., by at least about 30% or 50%), as measured by a visual analog scale (VAS) score or a numerical rating scale (NRS) score, for example.
9. The method of any one of the preceding embodiments, wherein the NK-1 antagonist is a selective NK-1 antagonist.
10. The method of any one of the preceding embodiments, wherein the NK-1 antagonist is selected from aprepitant (L-754030 or MK-(0)869), fosaprepitant (L-758298), befetupitant, burapitant (SSR-240600), casopitant (GW-679769), dapitant (RPR-100893), ezlopitant (C-11974), figopitant (BIIF-1149), lanepitant (LY-303870), maropitant (C-11972), netupitant, fosnetupitant, nolpitantium (SR-140333), orvepitant (GW-823296), rolapitant (SCH-619734), SCH-720881 (active metabolite of rolapitant), serlopitant (MK-(0)594 or VPD-737), tradipitant (VLY-686 or LY-686017), vestipitant (GW-597599), vofopitant (GR-205171), hydroxyphenyl propamidobenzoic acid, maltooligosaccharides (e.g., maltotetraose and maltopentaose), spantides (e.g., spantide I and II), AV-608, AV-818, AZD-2624, CGP-49823, CJ-17493, CP-96345, CP-99994, CP-122721, DNK-333 (NK1/NK2), FK-224 (NK1/NK2), FK-888, GR-82334, GR-203040, GR-205171, GSK-424887, HSP-117, KRP-103, L-703606, L-733060, L-736281, L-759274, L-760735, LY-686017, M516102, MDL-105212 (NK1/NK2), MK-0303 (L-001182885), MK-8478 (L-001983867), NKP-608, PD-154075, R-116031, R-116301, RP-67580, S-41744, SCH-206272 (NK-1/NK-2/NK-3), SCH-388714, SCH-900978, SLV-317, T-2328, TA-5538, TAK-637, TKA-731, WIN-51708, ZD-4974, ZD-6021 (NK1/NK2), and analogs, derivatives, prodrugs, metabolites, salts and stereoisomers thereof.
11. The method of any one of the preceding embodiments, wherein the NK-1 antagonist is aprepitant, fosaprepitant, rolapitant, orvepitant, serlopitant, or tradipitant, or a pharmaceutically acceptable salt, solvate, hydrate, clathrate, polymorph, prodrug, metabolite or stereoisomer thereof.
12. The method of any one of the preceding embodiments, wherein the NK-1 antagonist is serlopitant or a pharmaceutically acceptable salt, solvate, hydrate, clathrate, polymorph, prodrug, metabolite or stereoisomer thereof.
13. The method of any one of the preceding embodiments, wherein the NK-1 antagonist is not aprepitant or fosaprepitant.
14. The method of any one of the preceding embodiments, wherein the therapeutically effective amount (e.g., per day or per dose) of the NK-1 antagonist is about 0.25 or 1 to 200 mg, 0.25 or 1 to 150 mg, 0.25 or 1 to 100 mg, 0.25 or 1 to 50 mg, 0.25 or 1 to 10 mg, 10-20 mg, 20-30 mg, 30-40 mg, 40-50 mg, 50-100 mg, 100-150 mg or 150-200 mg.
15. The method of any one of the preceding embodiments, wherein the therapeutically effective amount of the NK-1 antagonist is administered one or more times a day, once every two days, once every three days, twice a week or once a week (e.g., once or twice daily).
16. The method of any one of the preceding embodiments, wherein the NK-1 antagonist is serlopitant, or a pharmaceutically acceptably salt or stereoisomer thereof, and the serlopitant or the pharmaceutically acceptably salt or stereoisomer thereof is administered at about 5 to 40 mg once a week.
17. The method of any one of the preceding embodiments, wherein the NK-1 antagonist is serlopitant, or a pharmaceutically acceptable salt or stereoisomer thereof, and the therapeutically effective amount of serlopitant or the pharmaceutically acceptable salt or stereoisomer thereof, is about 0.25 or 1 to 5 mg or 5-10 mg, or about 0.25 mg, 0.5 mg, 1 mg, 5 mg or 10 mg, once or twice daily.
18. The method of embodiment 17, wherein the therapeutically effective amount of serlopitant, or a pharmaceutically acceptable salt or stereoisomer thereof, is about 5 mg once daily.
19. The method of any one of the preceding embodiments, wherein the therapeutically effective amount of the NK-1 antagonist is administered over a period of at least about 1 week, 2 weeks, 3 weeks, 4 weeks, 5 weeks, 6 weeks, 2 months, 3 months, 4 months, 5 months, 6 months, 1 year, 2 years, 3 years, 4 years, 5 years or longer (e.g., at least about 6 weeks, 3 months, 6 months or 1 year).
20. The method of any one of the preceding embodiments, wherein the NK-1 antagonist is administered orally (e.g., as a tablet or capsule).
21. The method of any one of embodiments 1 to 19, wherein the NK-1 antagonist is administered parenterally (e.g., intravenously, subcutaneously or intramuscularly).
22. The method of any one of embodiments 1 to 19, wherein the NK-1 antagonist is administered topically (e.g., transdermally, transmucosally, pulmonarily, buccally or sublingually).
23. The method of any one of the preceding embodiments, wherein the NK-1 antagonist is serlopitant, or a pharmaceutically acceptable salt or stereoisomer thereof, and the serlopitant, or the pharmaceutically acceptable salt or stereoisomer thereof, is administered orally (e.g., as a tablet) in a dose of about 0.25, 0.5, 1, 5 or 10 mg (e.g., about 5 mg) once daily for at least about 1 week, 2 weeks, 3 weeks, 4 weeks, 5 weeks, 6 weeks, 2 months, 3 months, 4 months, 5 months, 6 months, 1 year, 2 years, 3 years, 4 years, 5 years or longer (e.g., at least about 6 weeks, 3 months, 6 months or 1 year).
24. The method of any one of the preceding embodiments, wherein at least one loading dose of the NK-1 antagonist is first administered, and a therapeutically effective maintenance dose of the NK-1 antagonist is subsequently administered.
25. The method of embodiment 24, wherein the therapeutically effective maintenance dose (e.g., per day or per dose) of the NK-1 antagonist is about 0.25 or 1 to 200 mg, 0.25 or 1 to 150 mg, 0.25 or 1 to 100 mg, 0.25 or 1 to 50 mg, 0.25 or 1 to 10 mg, 10-20 mg, 20-30 mg, 30-40 mg, 40-50 mg, 50-100 mg, 100-150 mg or 150-200 mg.
26. The method of embodiment 24 or 25, wherein the at least one loading dose of the NK-1 antagonist is about 1.5, 2, 3, 4 or 5 times (e.g., about 3 times) larger than the therapeutically effective maintenance dose of the NK-1 antagonist.
27. The method of any one of embodiments 24 to 26, wherein the therapeutically effective maintenance dose of the NK-1 antagonist is administered one or more times a day, once every two days, once every three days, twice a week or once a week (e.g., once or twice daily).
28. The method of any one of embodiments 24 to 27, wherein the NK-1 antagonist is serlopitant, or a pharmaceutically acceptable salt or stereoisomer thereof, and the therapeutically effective maintenance dose of serlopitant, or the pharmaceutically acceptable salt or stereoisomer thereof, is about 0.25 or 1 to 5 mg or 5-10 mg, or about 0.25 mg, 0.5 mg, 1 mg, 5 mg or 10 mg, once or twice daily, such as about 5 mg once daily.
29. The method of any one of embodiments 24 to 28, wherein the therapeutically effective maintenance dose of the NK-1 antagonist is administered over a period of at least about 1 week, 2 weeks, 3 weeks, 4 weeks, 5 weeks, 6 weeks, 2 months, 3 months, 4 months, 5 months, 6 months, 1 year, 2 years, 3 years, 4 years, 5 years or longer (e.g., at least about 6 weeks, 3 months, 6 months or 1 year).
30. The method of any one of embodiments 24 to 29, wherein the NK-1 antagonist is serlopitant, or a pharmaceutically acceptable salt or stereoisomer thereof, and the serlopitant, or the pharmaceutically acceptable salt or stereoisomer thereof, is administered in a loading dose of about 3-15 mg or 15-30 mg once or twice on day 1, followed by a maintenance dose of about 1-5 mg (e.g., about 1, 3 or 5 mg) or about 5-10 mg (e.g., about 5, 7.5 or 10 mg) once or twice daily for at least about 1 week, 2 weeks, 3 weeks, 4 weeks, 5 weeks, 6 weeks, 2 months, 3 months, 4 months, 5 months, 6 months, 1 year, 2 years, 3 years, 4 years, 5 years or longer (e.g., 15 mg on day 1 followed by 5 mg once daily for at least about 6 weeks, 3 months, 6 months or 1 year), where the loading dose is three times larger than the maintenance dose and serlopitant, or a pharmaceutically acceptable salt or stereoisomer thereof, is administered orally (e.g., as a tablet or capsule).
31. The method of any one of the preceding embodiments, wherein the NK-1 antagonist is administered at bedtime or in the morning.
32. The method of any one of the preceding embodiments, wherein the NK-1 antagonist is administered without food (e.g., at least about 1 or 2 hours before or after a meal, such as at least about 2 hours after an evening meal or at least about 2 hours before or after a meal in the morning).
33. The method of embodiment 1, wherein the pruritus is neuropathic itch, such as brachioradial pruritus or notalgia paresthetica.
34. The method of embodiment 33, wherein the pruritus is neurogenic itch.
35. The method of embodiment 33, wherein the pruritus is pruritus in the elderly (over 65 years of age).
36. The method of embodiment 33, wherein the pruritus is chronic idiopathic pruritus.
37. The method of any one of the preceding embodiments, wherein:
   i) the duration of the pruritus is about one year or longer;
   ii) the subject is about 40 years of age or older;
   iii) the subject has no inflammatory skin disease; and
   iv) the pruritus is idiopathic.
38. The method of any one of the preceding embodiments, wherein the pruritus is refractory or resistant to other antipruritic therapies without an NK-1 antagonist.
39. A method of treating a pruritus in a subject, comprising administering to the subject a therapeutically effective amount of a neurokinin-1 (NK-1) antagonist, wherein any one or more of i)-iv) apply:
   i) the duration of the pruritus is about one year or longer;
   ii) the subject is about 18 years of age or older;
   iii) the subject has no inflammatory skin disease at the time of administration; and
   iv) the pruritus is idiopathic.
40. The method of embodiment 39, wherein the NK-1 antagonist is aprepitant, fosaprepitant, rolapitant, orvepitant, serlopitant, or tradipitant, or a pharmaceutically acceptable salt or stereoisomer thereof.
41. The method of embodiment 39, wherein the NK-1 antagonist is serlopitant, or a pharmaceutically acceptable salt or stereoisomer thereof.
42. The method of embodiment 41, wherein the serlopitant, or the pharmaceutically acceptably salt or stereoisomer thereof, is administered once a week.
43. The method of any one of embodiments 39-42, wherein the NK-1 antagonist is administered orally in a tablet or capsule.
44. The method of any one of embodiments 39-43, wherein the subject is at least about 35 years of age, at least about 45 years of age, at least about 50 years of age, or at least about 60 years of age, at least about 70 years of age, or at least about 80 years of age.
45. The method of any one of embodiments 39-44, wherein all of i)-iv) apply.
46. A method of treating a pruritus in a subject, comprising administering to the subject a therapeutically effective amount of a neurokinin-1 (NK-1) antagonist, wherein any one or more of i)-iv) apply:
   i) the duration of the pruritus is about one year or longer;
   ii) the subject is about 40 years of age or older;
   iii) the subject has no inflammatory skin disease; and
   iv) the pruritus is of unknown origin.
47. The method of embodiment 46, wherein the subject has no inflammatory skin disease at the time of administration.
48. The method of embodiment 46, wherein the subject has no history of inflammatory skin disease.
49. The method of embodiment 46, wherein the subject has no inflammatory skin disease at the time of administration but has a history of inflammatory skin disease.
50. The method of embodiment 46, wherein the subject has no inflammatory skin disease at the time of administration and has no history of inflammatory skin disease.
51. The method of embodiments 46-50, wherein all of i)-iv) apply.
52. A kit comprising:
   a neurokinin-1 antagonist (e.g., serlopitant, or a pharmaceutically acceptable salt or stereoisomer thereof); and instructions for administering or using the neurokinin-1 antagonist to treat a pruritus in a subject, wherein any one or more of i)-iv) apply:
   i) the duration of the pruritus is about one year or longer;
   ii) the subject is about 40 years of age or older:
   iii) the subject has no inflammatory skin disease, and
   iv) the pruritus is idiopathic.
53. A kit of the embodiment 52, wherein all of i)-iv) apply.

### EXAMPLES

The following examples are intended only to illustrate the disclosure. Other assays, studies, protocols, procedures, methodologies, materials, substances, reagents and conditions may alternatively be performed or used as appropriate. All of the inactive pharmaceutical ingredients in the examples below comply with United States Pharmacopeia and The National Formulary requirements and are tested and released according to the monograph for each ingredient specified in the USP/NF compendium.

### Example 1. Preparation of Serlopitant Tablets

The NK-1 antagonist serlopitant can be formulated as a tablet for oral use. Table 1 shows qualitative/quantitative composition of exemplary dosages. Minor variations in the excipient quantities (+/−10%) may occur during the drug development process.

TABLE 1

| Components | Function | % of Composition |
|---|---|---|
| Serlopitant | Active agent | 1-6% |
| Microcrystalline cellulose | Diluent | 50-60% |
| Mannitol | Diluent | 20-30% |
| Croscarmellose Sodium | Disintegrant | 1-3% |
| Colloidal silica | Disintegrant | 0.25-0.5% |
| Sodium Lauryl Sulfate | Surfactant | 5-6% |
| Magnesium Stearate | Lubricant | 0.25-2% |

| | |
|---|---|
| Total Tablet Composition | 100% |

Tablet potencies of 0.25 mg, 1 mg and 5 mg are prepared as a compressed tablet formulation. The tablet manufacturing process is the same for all potencies. The process comprises the following steps: 1) serlopitant, mannitol and sodium lauryl sulfate are blended; 2) the remaining mannitol is added to the blender and mixed; 3) microcrystalline cellulose, croscartnellose sodium and colloidal silica are added to the blender containing the mixture above to complete the mixing, and the blend is de-agglomerated if necessary; 4) the blend is lubricated with magnesium stearate that has been previously screened, if necessary; 5) the lubricated blend is roller-compacted and milled, and then lubricated with magnesium stearate that has been previously screened, if necessary; and 6) the mixture is compressed into tablets of the appropriate weight.

### Example 2. Preparation of Serlopitant Capsules

Serlopitant can also be formulated as liquid-filled capsules. Table 2 shows qualitative/quantitative composition of exemplary dosages. Minor variations in the excipient quantities (41-10%) may occur during the drug development process.

TABLE 2

| | | Unit Strength | | | | | |
|---|---|---|---|---|---|---|---|
| Components | Function | 0.25 mg | | 1 mg | | 4 mg | |
| Capsule Fill | | | | | | | |
| Serlopitant | Active agent | 0.25 | mg | 1 | mg | 4 | mg |
| Mono- & Di-glycerides | Solubilizer | 399 | mg | 398.6 | mg | 395.6 | mg |
| Butylated Hydroxyanisole | Antioxidant | 0.40 | mg | 0.40 | mg | 0.40 | mg |
| Capsule Shell | | | | | | | |
| #0 White Opaque | Capsule shell | 96 | mg** | 96 | mg** | 96 | mg** |
| Hard Gelatin Capsule* | | | | | | | |

| | | | | |
|---|---|---|---|---|
| Gelatin*** | Banding | — | — | — |
| | component | | | |
| Polysorbate 80*** | Banding | — | — | — |
| | component | | | |

*Capsules are provided by Capsugel (Morristown, New Jersey) and contain gelatin and titanium dioxide
**Approximate weight of empty capsule shell
***As needed to seal the capsule shells

The formulation is prepared by dissolving the drug substance in mono- and di-glycerides. Furthermore, 0.1 wt % butylated hydroxyanisole is added as an antioxidant. Initial capsule strengths are dispensed into hard gelatin capsules and sealed by spraying with a 1:1 (wt/wt) water:ethanol solution. Subsequent potencies, including 0.25 mg, 1 mg and 4 mg, are dispensed into hard gelatin capsules and sealed with a band of gelatin/polysorbate 80. Corresponding placebo formulations are prepared in a similar manner, but without the addition of the drug substance and the antioxidant.

The capsule manufacturing process is the same for all potencies. The process comprises the following steps: 1) the mono- and di-glycerides are melted at 40° C., if necessary; 2) the mono- and di-glycerides are added to an appropriately sized, jacketed vessel and mixing is initiated; 3) the butylated hydroxyanisole is added to the mono- and di-glycerides and mixed until dissolved (minimum of 10 min); 4) serlopitant is slowly added to the mixture and mixed until dissolved (visual confirmation); 5) the solution is filled into hard gelatin capsules; 6) the filled capsules are sealed with a mixture of gelatin and polysorbate 80; 7) the sealed capsules are allowed to dry overnight and then the capsules are visually inspected for leaking; 8) the acceptable capsules may be weight-sorted, if necessary; and 9) the finished product is packaged in appropriate containers.

### Example 3. Clinical Study of Serlopitant for Prurigo Nodularis (PN)

A well-controlled human clinical trial assessing the efficacy of serlopitant in the treatment of subjects with Prurigo Nodularis (PN) was approved by an Institutional Review Board and was conducted in accordance with the International Conference on Harmonisation (ICH) Guidelines for Good Clinical Practices, the U.S. Code of Federal Regulations, the Health Insurance Portability and Accountability Act (HIPAA), and any local regulatory requirements. The study was a Phase 11 randomized, double-blind, parallel-group, placebo-controlled, multicenter trial designed to evaluate the efficacy and safety of serlopitant (5 mg) versus placebo in subjects with PN.

Prurigo Nodularis (PN) is a chronic condition characterized by a severely itching papulonodular eruption due to chronic scratching along with chronic pruritus of unknown aetiology. The study subject population was adult males and females 18-80 years of age who have PN of more than 6 weeks in duration that is unresponsive or inadequately responsive to topical glucocorticioid or antihistamine therapies, and with a baseline Visual Analog Scale (VAS) pruritus score ≥70.

Subjects were randomized to receive serlopitant (5 mg) or placebo in a 1:1 randomization. Subjects received a loading dose of 3 tablets on Day 1 followed by 1 tablet per day for 8 weeks. The maximum study duration for each subject was about 14 weeks and included a screening period of up to 4 weeks, a treatment period of 8 weeks, and a follow-up period of 2 weeks. The study parameters are summarized in Table 3,

TABLE 3

| | |
|---|---|
| Study Title | A Randomized, Double-Blind, Parallel-Group, Placebo- |
| | Controlled Study of Serlopitant in Subjects with Prurigo |
| | Nodularis |
| Development Phase | Phase II |
| Study Objectives | The primary objectives of this study are to evaluate the efficacy |
| | and safety of Serlopitant 5-mg tablets and the placebo taken |
| | orally once daily for 8 weeks for the treatment of prurigo |
| | nodularis (PN). |
| Study Design | Randomized, double-blind, parallel-group, placebo-controlled |
| Sample Size | Approximately 140 subjects who meet the entry criteria will be |
| | randomly assigned in a 1:1 ratio to receive Serlopitant or |
| | placebo . . . Subjects received a loading dose of 3 tablets on Day 1 |
| | followed by 1 tablet per day for 8 weeks. |
| Study Population | Men and women 18 to 80 years of age who have PN of more |
| | than 6 weeks duration that is unresponsive or inadequately |
| | responsive to topical glucocorticoid or antihistamine therapies, |
| | and with a baseline Visual Analog Scale (VAS) pruritus score ≥ |
| | 70. Subjects with creatinine of >2.4 mg/dL or malignancy |
| | secondary involving the skin will be excluded. Subjects with |
| | suspicion of drug-induced PN will also be excluded. |
| Investigational Product | Oral daily tablets of Serlopitant (5 mg) |
| Dosage and Frequency | Serlopitant: Loading dose of three 5-mg tablets on Day 1 followed by |
| | 1 tablet once daily by mouth for 8 weeks. |
| | Matching placebo: Loading dose of 3 placebo tablets on Day 1 |
| | followed by 1 tablet once daily by mouth for 8 weeks. |
| Reference Product | None |
| Control Product | Matching tablets of placebo |
| Efficacy Evaluation | Primary: The primary endpoints of this double-blind, |
| Criteria | randomized |
| | clinical trial are pairwise comparisons between treatments of |
| | the VAS score (average) over 24 hour period and the safety and |
| | tolerability of Serlopitant 5-mg tablets and the placebo taken |
| | orally once daily for 8 weeks for the treatment of prurigo |
| | nodularis (PN). |
| | Secondary: The secondary endpoints are descriptive pairwise |
| | comparisons between the Serlopitant and placebo for the |
| | following: |
| | Mean change from Baseline in VRS, VAS (worst), patient |
| | global assessment score, dynamic score, and NRS. |
| | Mean change from Baseline in Quality of Life questionnaire |
| | results (Dermatology Life Quality Index (DLQI)/pruritus |
| | specific Quality of Life (ItchyQoL)). The DLQI/ItchyQoL will |
| | be scored according to the instrument developers rules; |
| | Mean change from Baseline in the Patient Benefit Index, |
| | Version for Patients with Pruritus (PBI-P) questionnaire |
| | results; |
| | Mean change from Baseline in PN skin lesions as measured |
| | by the Prurigo Activity Score (PAS): |
| | Mean change from Baseline in the Investigator Global |
| | Assessment (IGA); |
| | Time course of changes in VRS, NRS and VAS scores. |
| | Percentage of patients in each group requiring rescue |
| | medication with loratadine or ceterizine. |
| Safety Evaluation | Safety was assessed by adverse events, serious adverse events, |
| Criteria | electrocardiograms, vital signs, abbreviated physical |
| | examinations, and blood and urine laboratory tests. |
| Statistical Methods | Primary efficacy analysis will be conducted on the intent to |
| | treat population. |
| | The efficacy endpoint will be assessed by the pairwise |
| | estimated difference in VAS score between active and placebo groups. |
| Study Sites | Multiple sites in the United States |

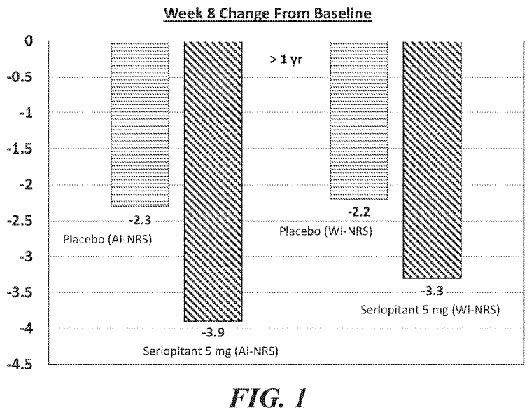

FIG. 1 shows the effects of serlopitant for subjects who had pruritus of duration one year or longer as measured by change in AI-NRS and WI-NRS from baseline at week 8.

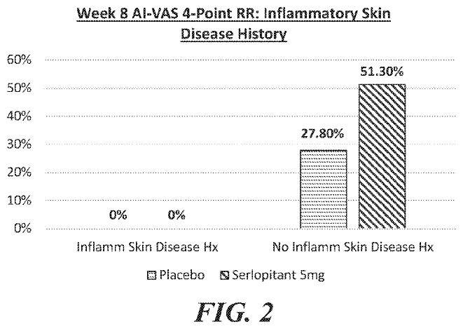

FIG. 2 shows the effects of serlopitant for subjects with inflammatory skin disease and for subjects with no inflammatory skin disease. For subjects with inflammatory skin disease, the conditions are shown in Table 4.

TABLE 4

| | Placebo | Serlopitant (5 mg) |
|---|---|---|
| Condition | (N = 63) | (N = 64) |
| Atopic dermatitis | 4 | 3 |
| Dermatitis | 1 | 0 |
| Dermatosis | 0 | 1 |
| Eczema nummular | 0 | 1 |
| Lichen planus | 0 | 1 |
| Pemphigoid | 0 | 1 |
| Psoriasis | 1 | 0 |

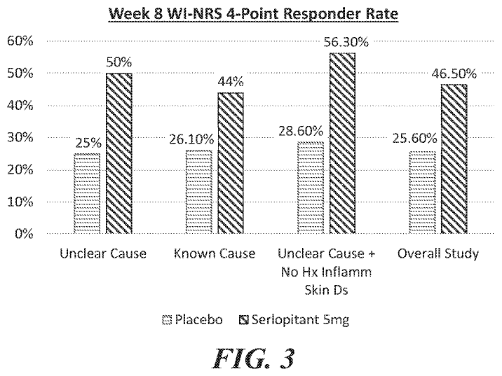

FIG. 3 shows the effects of serlopitant for subjects with varying known causes or unclear cause of PN as measured by percent improvement in WI-NRS from baseline at week 8. The causes of PN for these subjects are shown in Table 5.

TABLE 5

| | Placebo | Serlopitant (5 mg) |
|---|---|---|
| Cause for PN | (N = 63) | (N = 64) |
| Atopic diathesis | 26 | 29 |
| Dermatosis | 0 | 2 |
| Liver disease | 0 | 2 |
| Systemic disease | 7 | 3 |
| Unclear | 27 | 29 |

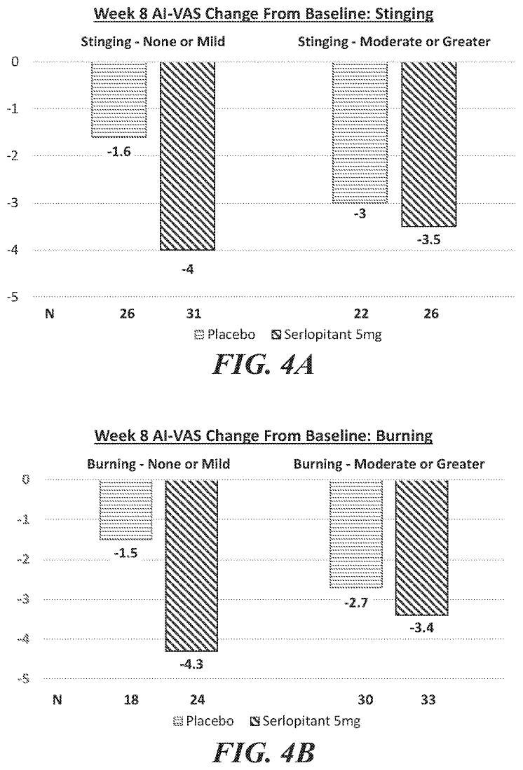

FIGS. 4A and 4B show the effects of serlopitant on patients with various stinging (A) or burning (B) conditions as measured by change in AI-VAS from baseline at week 8.

### Example 4. Clinical Study of Serlopitant for Pruritus in Subjects with History of Atopic Dermatitis

A well-controlled human clinical trial assessing the efficacy of serlopitant in the treatment of pruritus was approved by an Institutional Review Board and was conducted in accordance with the ICH Guidelines for Good Clinical Practices, German regulations on recordkeeping of subject information, and any local regulatory requirements. The study was a Phase 11 randomized, double-blind, placebo-controlled, multicenter trial designed to evaluate the efficacy and safety of serlopitant versus placebo in subjects with pruritus. The study subject population was males and females 13 years of age or older who had pruritus for at least 4 weeks duration prior to initial screening visit and had a history of atopic dermatitis.

Approximately 484 adult and adolescent subjects with pruritus and a history of atopic dermatitis were randomized to receive one of the three dose groups in a 1:1:1 randomization. Subjects received 1 mg or 5 mg Serlopitant tablet or a placebo tablet once daily by mouth for 6 weeks, following a 3-tablet loading dose on a first day of the treatment period. The primary efficacy endpoint was itch severity as measured on a WI-NRS, summarized as a percentage change from baseline. The key secondary efficacy endpoint WI-NRS 4-point responder rates at Week 6 were also recorded. The maximum study duration for each subject was about 12 weeks and included a screening period of up to 2 weeks, a treatment period of 6 weeks, and a follow-up period of 4 weeks. The study parameters are summarized in Table 6.

TABLE 6

| | |
|---|---|
| Study Title | A Randomized, Double-Blind, Placebo-Controlled Study of |
| | Serlopitant in Subjects with a History of Atopic Dermatitis |
| Development Phase | Phase 11 |
| Study Objectives | Evaluate the efficacy and safety of serlopitant in subjects with a |
| | history of atopic dermatitis |
| Study Design | Randomized, double-blind, placebo-controlled |
| Sample Size | Approximately 484 adult and adolescent subjects were |
| | randomized to receive one of the three dose groups in a 1:1:1 |
| | randomization (Serlopitant 1 mg, or 5 mg vs. placebo) for 6 |
| | weeks |
| Study Population | The subjects were male and female 13 years of age or older who |
| | had pruritus of at least 4 weeks duration prior to the initial |
| | Screening visit and who had a history of atopic dermatitis. |
| | Subject had Worst-Itch Numeric Rating Scale (WI-NRS) score ≥ |
| | 7 in the 24-hour period prior to the Screening visit. |
| | Subject had average weekly WI-NRS score ≥ 6 for each week of |
| | the Screening period, as recorded in the eDiary. |
| | Subject had diagnosis of atopic dermatitis, as defined by the |
| | 2014 American Academy of Dermatology (AAD) Guidelines of |
| | Care for the Management of Atopic Dermatitis. |
| | Subject did not receive prior neurokinin-1 receptor antagonists |
| | (such as Serlopitant) treatment. |
| Investigational Product | Oral daily tablet of Serlopitant |
| Dosage and Frequency | Serlopitant: 1 mg or 5 mg once daily by mouth for 6 weeks, |
| | following a 3-tablet loading dose on the first day of the treatment |
| | period. |
| | Matching placebo: Once daily by mouth for 6 weeks, following a |
| | 3-tablet loading dose on the first day of the treatment period. |
| Reference Product | None |
| Control Product | Matching placebo |
| Efficacy Evaluation | The primary efficacy endpoint was the WI-NRS from baseline to |
| Criteria | Week 6. |
| | Secondary efficacy endpoints included analyses of WI-NRS 4- |
| | point responder rate at Week 6 and change in ItchyQoL from |
| | baseline to Week 6. |
| Safety Evaluation | Safety was assessed by adverse events, serious adverse events, |
| Criteria | electrocardiograms, vital signs, abbreviated physical |
| | examinations, and blood and urine laboratory tests. |
| Statistical Methods | Efficacy analyses will be based upon an intent-to-treat |
| | philosophy. The primary efficacy population will be the Full |
| | Analysis Set (FAS) that will include all randomized and treated |
| | subjects. Subjects will be analyzed within the treatment group to |
| | which they are randomized. |
| | Efficacy Analyses: |
| | The primary efficacy endpoint will be tested using an analysis of |
| | variance (ANOVA) model controlling for the stratification |
| | factors. A main effects model with an interaction term (treatment |
| | by stratification factors) and a Type II hypothesis will be used. |
| | Missing data imputation will be used for subjects who fail to |
| | complete the eDiary at Week 6 or receive excluded therapy. The |
| | primary endpoint will be summarized with descriptive statistics |
| | by treatment group and study week. These summary statistics |
| | will include estimates of the treatment differences and associated |
| | confidence interval. |
| | Testing, using an analysis of variance (ANOVA) model or |
| | Cochran Mantel Haenszel (CMH) test of the key secondary |
| | endpoints will also be employed. |
| Study Sites | Multiple sites in the United States |

### Example 5. Clinical Study of Serlopitant for Chronic Pruritus

A well-controlled human clinical trial assessing the efficacy of serlopitant in the treatment of chronic pruritus is conducted in accordance with the ICH Guidelines for Good Clinical Practices, the U.S. Code of Federal Regulations. HIPAA and any local regulatory requirements. The study is a Phase II randomized, double-blind, placebo-controlled, multicenter trial designed to evaluate the efficacy, tolerability and safety of serlopitant versus placebo in subjects with idiopathic pruritus. The study subject population includes adult males and females 18-65 years of age. The subjects had ongoing pruritus of at least 6 weeks duration that is unresponsive or inadequately responsive to topical glucocorticoid and antihistamine therapies.

Approximately 264 adult subjects 18-65 years of age with pruritus were randomized to receive one of the four dose groups in a 1:1:1:1 randomization (Serlopitant 0.25 mg, 1 mg, or 5 mg vs. placebo). Subjects received a loading dose of 3 tablets on Day 1 and thereafter received 1 tablet per day for 6 weeks. The efficacy endpoint was percent change in VAS from baseline. The maximum study duration for each subject was about 12 weeks and included a screening period of up to 2 weeks, a treatment period of 6 weeks, and a follow-up period of 4 weeks. The study parameters are summarized in Table 7.

TABLE 7

| | |
|---|---|
| Study Title | A Randomized, Double-Blind, Placebo-Controlled Study of |
| | Serlopitant in Subjects with Chronic Idiopathic Pruritus |
| Development Phase | Phase II |
| Study Objectives | Assess the efficacy and safety of serlopitant in treating chronic |
| | pruritus |
| Study Design | Randomized, double-blind, placebo-controlled study |
| Sample Size | Approximately 264 adult subjects were randomized to receive |
| | one of the four dose groups in a 1:1:1:1 randomization |
| | (Serlopitant 0.25 mg, 1 mg, or 5 mg vs. placebo) for 6 weeks. |
| Study Population | The subjects were male and female 18 to 65 years old. Subjects |
| | will include those with chronic pruritus of at least 6 weeks |
| | duration that is unresponsive or inadequately responsive to |
| | topical glucocorticoid and antihistamine therapies, and with a |
| | baseline Visual Analog Scale (VAS) pruritus score ≥ 7. Subjects |
| | with chronic renal or hepatic disease or malignancy involving the |
| | skin will be excluded. Subjects on medications known to cause |
| | pruritus will also be excluded, who had ongoing pruritus of |
| | unknown etiology for at least one year, involving body surface |
| | areas of at least two different sensory dermatomes. |
| Investigational Product | Oral daily tablet of serlopitant |
| Dosage and Frequency | Serlopitant: 0.25 mg, 1 mg, or 5 mg once daily by mouth |
| | following a 3-tablet loading dose on Day 1. |
| | Matching placebo: Once daily by mouth, following a 3-tablet |
| | loading dose on Day 1. |
| Reference Product | None |
| Control Product | Matching placebo |
| Efficacy Evaluation | The primary endpoint will be the percent change in VAS from |
| Criteria | Baseline, comparing drug to placebo for each dose group. |
| | Secondary endpoints will be Subject Global Assessment, |
| | Dermatology Life Quality Index (DLQI), photographs of lesions |
| | (for illustrative purposes only), Pittsburgh Insomnia Symptom |
| | Questionnaire (PSSQ_I), and the Physicians Global |
| | Assessment. |
| Safety Evaluation | The intensity, duration and causal relationship to the |
| Criteria | investigational product are to be rated for all adverse events. |
| | Laboratory assessments, vital signs, physical examinations, |
| | electrocardiograms (ECGs), and concentration of drug product at |
| | trough levels will also be used to assess safety. |
| Statistical Methods | The primary analysis will be conducted on an Intent-to-Treat |
| | (ITT) population. |
| Study Sites | Multiple sites in the United States |

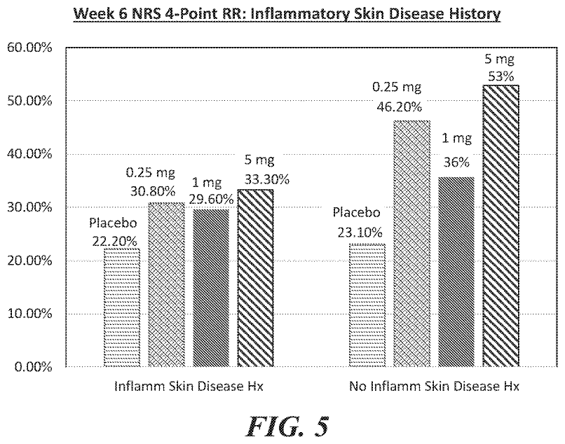

FIG. 5 shows the effects of serlopitant on subjects with inflammatory skin disease compared to subjects without inflammatory skin disease as measured by WI-NRS 4-Point Responder Rate at Week 6. Results in FIG. 5 show that patients without a medical history of inflammatory skin disease exhibited treatment group response rates of approximately 36% to 53% compared with a placebo group response of 23%. This corresponds to treatment group response rates of approximately 30% to 33% and placebo group response of 22% in patients that had a medical history of inflammatory skin disease.

Table 3 below shows the results of the post hoc analysis of WI-NRS 4-Point Responder Rate at Week 6.

TABLE 8
WI-NRS 4-point Responder Rate at Week 6

| | Patients without | Patients with | |
|---|---|---|---|
| | inflammatory | inflammatory | |
| | skin disease | skin disease | All patients |
| Placebo | 23.1% (n = 26) | 22.2% (n = 27) | 22.6% (n = 53) |
| 0.25 mg | 46.2% (n = 26) | 30.8% (n = 26) | 38.5% (n = 52) |
| 1 mg | 35.7% (n = 28) | 29.6% (n = 27) | 32.7% (n = 55) |
| 5 mg | 52.9% (n = 34) | 33.3% (n = 18) | 46.2% (n = 52) |

These results suggest that patients without inflammatory skin disease appeared to respond better to serlopitant therapy than patients with inflammatory skin disease.

### Example 6: Age-Related Effects of Serlopitant

Data obtained from the clinical studies described in Examples 6-8 for patients administered 1 mg and 5 mg serlopitant were pooled and evaluated on the basis of the age of the subjects.

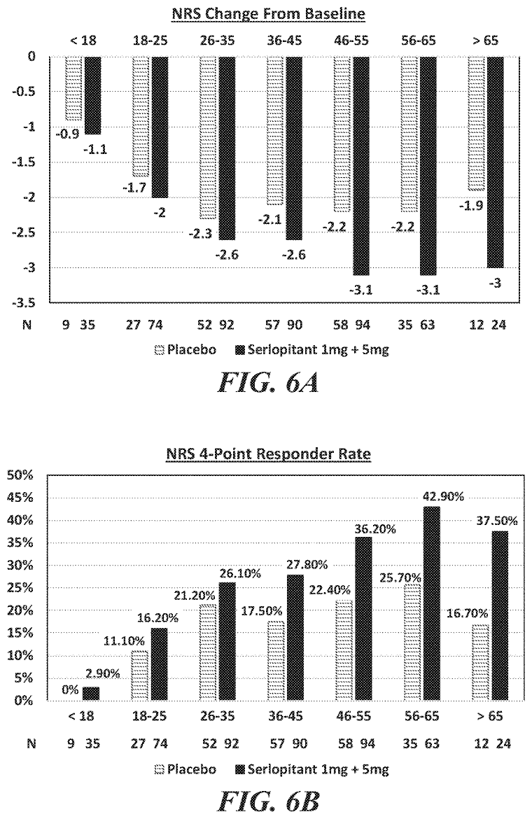

FIG. 6A shows the effect of serlopitant as function of age as measured by NRS change from baseline. FIG. 6B shows the effect of serlopitant as a function of age as measured by NRS 4-point Responder Rate percentage change from baseline. These results suggest that older patients (e.g., 40 years or older) responded better to serlopitant therapy than patients who were younger.

Additional or different clinical trials according to a similar study design can be conducted to study, e.g., different dosages (e.g., about 1 mg or 10 mg once daily), different dosing schedules (e.g., about 1 mg or 5 mg twice daily) or different modes of administration (e.g., oral inhalation) of serlopitant, or different lengths of treatment (e.g., about 2 months, 3 months, 6 months or 1 year) with serlopitant. Furthermore, the efficacy of serlopitant in specific subject populations, such as children, adolescents, the elderly (e.g., at least 45 years old, at least 50 years old, at least 60 years old, at least 70 years old, or at least 80 years old), and in treating pruritus of a longer duration (e.g., at least 6 months, 1 year, 2 years, 3 years or 5 years) can be determined in additional or different clinical trials conducted in a similar fashion.

It is understood that, while particular embodiments have been illustrated and described, various modifications may be made thereto and are contemplated herein. It is also understood that the disclosure is not limited by the specific examples provided herein. The description and illustration of embodiments and examples of the disclosure herein are not intended to be construed in a limiting sense. It is further understood that all aspects of the disclosure are not limited to the specific depictions, configurations or relative proportions set forth herein, which may depend upon a variety of conditions and variables. Various modifications and variations in form and detail of the embodiments and examples of the disclosure will be apparent to a person skilled in the art. It is therefore contemplated that the disclosure also covers any and all such modifications, variations and equivalents.

1. A method of treating a pruritus in a subject, comprising administering to the subject a therapeutically effective amount of 3-[(3aR,4R,5S,7aS)-5-[(1R)-1-[3,5-bis(trifluoromethyl)phenyl]ethoxy]-4-(4-fluorophenyl)-1,3,3a,4,5,6,7,7a-octahydroisoindol-2-yl]cyclopent-2-en-1-one (serlopitant) or a pharmaceutically acceptable salt, solvate, hydrate, clathrate, polymorph, prodrug, metabolite or stereoisomer thereof, wherein:
i) the duration of the pruritus is about six months or longer;
ii) the subject is about 40 years of age or older;
iii) the subject has no inflammatory skin disease; and
iv) the pruritus is idiopathic.

2. The method of claim 1, wherein the pruritus has duration of at least about 1 year, 2 years, 3 years, 5 years, 7 years, 10 years or 15 years.

3. The method of claim 1 or 2, wherein the pruritus is characterized by spontaneous itch, alloknesis or hyperknesis, or any combination or all thereof.

4. The method of claim 3, wherein serlopitant or the pharmaceutically acceptable salt, solvate, hydrate, clathrate, polymorph, prodrug, metabolite or stereoisomer thereof reduces the frequency or/and the intensity of spontaneous itch, alloknesis or hyperknesis, or any combination or all thereof, by at least about 20%, 30%, 40%, 50%, 60%, 70%, 80%, 90% or 95%, as measured by a visual analog scale (VAS) score or a numerical rating scale (NRS) score.

5. The method of any one of the preceding claims, wherein the therapeutically effective amount of serlopitant or the pharmaceutically acceptable salt, solvate, hydrate, clathrate, polymorph, prodrug, metabolite or stereoisomer thereof is administered one or more times a day, once every two days, once every three days, twice a week or once a week.

6. The method of any one of the preceding claims, wherein the therapeutically effective amount of serlopitant or the pharmaceutically acceptable salt, solvate, hydrate, clathrate, polymorph, prodrug, metabolite or stereoisomer thereof is about 0.25 or 1 to 5 mg or 5-10 mg, or about 0.25 mg, 0.5 mg, 1 mg, 5 mg or 10 mg, once or twice daily.

7. The method of any one of claims 1-5, wherein the therapeutically effective amount of serlopitant or the pharmaceutically acceptable salt, solvate, hydrate, clathrate, polymorph, prodrug, metabolite or stereoisomer thereof antagonist is about 5 mg once daily.

8. The method of any one of claims 1-5, wherein the therapeutically effective amount of serlopitant or the pharmaceutically acceptable salt, solvate, hydrate, clathrate, polymorph, prodrug, metabolite or stereoisomer thereof is about 5 to 40 mg once a week.

9. The method of any one of the preceding claims, wherein the therapeutically effective amount of serlopitant or the pharmaceutically acceptable salt, solvate, hydrate, clathrate, polymorph, prodrug, metabolite or stereoisomer thereof is administered over a period of at least about 1 week, 2 weeks, 3 weeks, 4 weeks, 5 weeks, 6 weeks, 2 months, 3 months, 4 months, 5 months, 6 months, 1 year, 2 years, 3 years, 4 years, or 5 years.

10. The method of any one of the preceding claims, wherein serlopitant or the pharmaceutically acceptable salt, solvate, hydrate, clathrate, polymorph, prodrug, metabolite or stereoisomer thereof is administered orally, parenterally or topically.

11. The method of any one of the preceding claims, wherein serlopitant or the pharmaceutically acceptable salt, solvate, hydrate, clathrate, polymorph, prodrug, metabolite or stereoisomer thereof is administered orally in a dose of about 0.25, 0.5, 1, 5 or 10 mg once daily for at least about 1 week, 2 weeks, 3 weeks, 4 weeks, 5 weeks, 6 weeks, 2 months, 3 months, 4 months, 5 months, 6 months, 1 year, 2 years, 3 years, 4 years, or 5 years.

12. The method of any one of the preceding claims, wherein at least one loading dose of serlopitant or the pharmaceutically acceptable salt, solvate, hydrate, clathrate, polymorph, prodrug, metabolite or stereoisomer thereof is first administered, and a therapeutically effective maintenance dose of serlopitant or the pharmaceutically acceptable salt, solvate, hydrate, clathrate, polymorph, prodrug, metabolite or stereoisomer thereof is subsequently administered.

13. The method of claim 12, wherein the at least one loading dose of serlopitant or the pharmaceutically acceptable salt, solvate, hydrate, clathrate, polymorph, prodrug, metabolite or stereoisomer thereof is about 1.5, 2, 3, 4 or 5 times larger than the therapeutically effective maintenance dose.

14. The method of claim 12 or 13, wherein the therapeutically effective maintenance dose of serlopitant or the pharmaceutically acceptable salt, solvate, hydrate, clathrate, polymorph, prodrug, metabolite or stereoisomer thereof is about 0.25 or 1 to 5 mg or 5-10 mg, or about 0.25 mg, 0.5 mg, 1 mg, 5 mg or 10 mg, once or twice daily.

15. The method of any one of claims 12-14, wherein serlopitant or the pharmaceutically acceptable salt, solvate, hydrate, clathrate, polymorph, prodrug, metabolite or stereoisomer thereof is administered in a loading dose of about 3-15 mg or 15-30 mg once or twice on day 1, followed by a maintenance dose of about 1-5 mg or about 5-10 mg once or twice daily for at least about 1 week, 2 weeks, 3 weeks, 4 weeks, 5 weeks, 6 weeks, 2 months, 3 months, 4 months, 5 months, 6 months, 1 year, 2 years, 3 years, 4 years, or 5 years, where the loading dose is three times larger than the maintenance dose and serlopitant or the pharmaceutically acceptable salt, solvate, hydrate, clathrate, polymorph, prodrug, metabolite or stereoisomer thereof is administered orally.

16. The method of any one of the preceding claims, wherein serlopitant or the pharmaceutically acceptable salt, solvate, hydrate, clathrate, polymorph, prodrug, metabolite or stereoisomer thereof is administered at bedtime or in the morning.

17. The method of any one of the preceding claims, wherein serlopitant or the pharmaceutically acceptable salt, solvate, hydrate, clathrate, polymorph, prodrug, metabolite or stereoisomer thereof is administered without food at least about 2 hours before or after a meal.

18. The method of any one of claims 1-17, wherein the pruritus is chronic idiopathic pruritus.

19. Serlopitant or a pharmaceutically acceptable salt, solvate, hydrate, clathrate, polymorph, prodrug, metabolite or stereoisomer thereof for use in the treatment of a pruritus in a subject, wherein:
i) the duration of the pruritus is about six months or longer;
ii) the subject is about 40 years of age or older;
iii) the subject has no inflammatory skin disease; and
iv) the pruritus is idiopathic.

20. A composition comprising serlopitant or a pharmaceutically acceptable salt, solvate, hydrate, clathrate, polymorph, prodrug, metabolite or stereoisomer thereof for use in the treatment of a pruritus in a subject, wherein:
i) the duration of the pruritus is about six months or longer;
ii) the subject is about 40 years of age or older;
iii) the subject has no inflammatory skin disease, and
iv) the pruritus is idiopathic.

21. Use of serlopitant or a pharmaceutically acceptable salt, solvate, hydrate, clathrate, polymorph, prodrug, metabolite or stereoisomer thereof in the preparation of a medicament for the treatment of a pruritus in a subject, wherein:
i) the duration of the pruritus is about six months or longer;
ii) the subject is about 40 years of age or older;
iii) the subject has no inflammatory skin disease; and
iv) the pruritus is idiopathic.

22. A kit comprising:
serlopitant or a pharmaceutically acceptable salt, solvate, hydrate, clathrate, polymorph, prodrug, metabolite or stereoisomer thereof; and
instructions for administering or using serlopitant or the pharmaceutically acceptable salt, solvate, hydrate, clathrate, polymorph, prodrug, metabolite or stereoisomer thereof to treat a pruritus in a subject, wherein:
i) the duration of the pruritus is about six months or longer:
ii) the subject is about 40 years of age or older;
iii) the subject has no inflammatory skin disease; and
iv) the pruritus is idiopathic.
